(19) United States
(12) Patent Application Publication
(10) Pub. No.: US 2008/0085279 A1
(43) Pub. Date: Apr. 10, 2008

(54) Cancer Treatment

(75) Inventors: Mark Thomas Boyd, Liverpool (GB); Nikolina Vlatkovic, Liverpool (GB); Mark Brady, Liverpool (GB)

Correspondence Address: DAVIS WRIGHT TREMAINE LLP/Los Angeles, 865 FIGUEROA STREET, SUITE 2400, LOS ANGELES, CA, 90017-2566, US

(73) Assignee: University of Liverpool, Liverpool (GB)

(21) Appl. No.: 11/793,698

(22) PCT Filed: Dec. 22, 2005
(86) PCT No.: PCT/GB05/05010

§ 371 Date: Jun. 21, 2007

(30) Foreign Application Priority Data
Dec. 23, 2004 (GB) 0428187.9

(51) Int. Cl. A61K 31/70; A61K 39/395; A61P 43/00; C12Q 1/68; G01N 33/574

(52) U.S. Cl. 424/139.1; 424/152.1; 424/172.1; 435/6; 435/7.23; 514/44

(57) ABSTRACT

The present invention relates to the inhibitors of MDM2 Binding Protein (MTBP) activity that may be used as medicaments. Such medicaments may be used to prevent or treat cancers. A preferred inhibitor is an siRNA molecule that is specific for silencing MTBP expression. The invention further relates to screening methods (e.g. for identifying MTBP inhibitors that may be used to treat cancers).

The present invention relates to the treatment of cancer.

Many different forms of cancer exist, and it is believed that there are many different causes of the disease. The incidence of cancer varies, but it represents the second highest cause of mortality, after heart disease, in most developed countries. Current estimates suggest that 33% of Americans alive at present will suffer from some form of cancer. Methods of treatment for cancer exist, although there is a well recognised need to develop new and improved techniques.

p53 is a protein that is well known to be associated with carcinogenesis. It is a critical co-ordinator of a wide range of cellular stresses ranging from myocyte stretch-induced apoptosis to increased global DNA repair in fibroblasts exposed to UV. To facilitate a rapid response to stress, cells have evolved a mechanism that relies upon stabilisation and activation by post-translational modification of existing constitutively expressed p53 protein. In normal cells it has been found that p53 is both functionally inhibited and moreover, maintained in an unstable state by the action of MDM2.

Originally discovered as one of three genes amplified on double minute chromosomes in a tumourigenic derivative of NIH 3T3 cells, MDM2 was later shown to possess oncogenic potential when over-expressed and to confer tumourigenic potential upon non-transformed rodent fibroblasts in athymic nude mice. MDM2 can immortalise rat embryo fibroblasts and can co-operate with activated RAS to transform these cells. Elevated levels of MDM2 protein have been found in a variety of human tumours, most notably in soft tissue sarcomas where up to 30% of primary tumours contain multiple copies of the MDM2 gene. One mechanism by which MDM2 over-expression promotes tumour development is through its ability to bind to the p53 tumour suppressor, thereby blocking the transactivation, cell cycle arrest and apoptotic functions of p53. MDM2 can inhibit p53 activity in a number of ways including preventing p53 from recruiting TAFs, promoting nuclear export, inhibiting p53 acetylation, and perhaps most importantly by virtue of its function as an E3 ubiquitin ligase with specificity for, amongst others, p53. In addition to regulating p53 levels by targeting p53 for proteasomal degradation, MDM2 also transfers ubiquitin to itself, MDMX, the β2 adrenergic receptor, glucocorticoid receptor, TIP60 and PCAF.

Induction of p53's transcriptional activity leads, inter alia, to increases in MDM2 mRNA and MDM2 protein, and thus an auto-regulatory feedback loop exists between these two proteins. The importance of this feedback loop has been confirmed by studies of transgenic animals. Inactivation of the p53 tumour-suppressor protein is a key event in carcinogenesis, as illustrated by the fact that more than 50% of all human malignancies harbour mutations of the p53 gene. It has been found that the p53 gene is rarely mutated in primary tumours (especially sarcomas) in which the MDM2 gene is amplified, although there is increasingly good evidence of exceptions to this in carcinomas. Thus MDM2 over-expression blocks p53 function in vivo and this contributes to the development of tumours. Together, these results demonstrate that a primary function of MDM2, at least during development, is to regulate p53 function.

It will therefore be appreciated that investigating the interaction between MDM2 and p53 may provide useful insights into carcinogenesis. Recent research, and indeed the inventors own preliminary work (see WO 02/04601) investigated the effect on MDM2 activity of the MDM2 Binding Protein (MTBP). MTBP was originally identified as part of a systematic search for MDM2 binding proteins and was understood to induce an arrest in the cell cycle and thereby stop cell growth. Accordingly MTBP was reported to be tumour suppressive (see page 1 of WO 02/04601).

It is an objective of the present invention, to overcome the problems associated with the prior art and provide new medicaments for use in cancer therapy.

According to a first aspect of the present invention, there is provided an inhibitor of MDM2 Binding Protein (MTBP) activity for use as a medicament.

According to a second aspect of the invention, there is provided the use of an inhibitor of MDM2 Binding Protein (MTBP) activity in the manufacture of a medicament for the treatment of cancer.

According to a third aspect of the present invention, there is provided a method of treating or preventing cancer comprising administering to a subject in need of such treatment a therapeutically effective amount of an inhibitor of MDM2 Binding Protein (MTBP) activity.

Human MTBP is an approximately 102 kDa protein of 904 amino acids. Human MTBP has been cloned and sequenced. The protein sequence (SEQ ID No.1) is shown below:—

MDRYLLLVIWGEGKFPSAASREAEHGPEVSSGEGTENQPDFTAANVYHLL
KRSISASINPEDSTFPACSVGGIPGSKKWFFAVQAIYGFYQFCSSDWQEI
HFDTEKDKIEDVLQTNIEECLGAVECFEEEDSNSRESLSLADLYEEAAEN
LHQLSDKLPAPGRAMVDIILLLSDKDPPKLKDYLPTVGALKHLREWYSAK
ITIAGNHCEINCQKIAEYLSANVVSLEDLRNVIDSKELWRGKIQIWERKF
GFEISFPEFCLKGVTLKNFSTSNLNTDFLAKKIIPSKDKNILPKVFHYYG
PALEFVQMIKLSDLPSCYMSDIEFELGLTNSTKQNSVLLLEQISSLCSKV
GALFVLPCTISNILIPPPNQLSSRKWKEYIAKKPKTISVPDVEVKGECSS
YYLLLQGNGNRRCKATLIHSANQINGSFALNLIHGKMKTKTEEAKLSFPF
DLLSLPHFSGEQIVQREKQLANVQVLALEECLKRRKLAKQPETVSVAELK
SLLVLTRKHFLDYFDAVIPKMILRKMDKIKTFNILNDFSPVEPNSSSLME
TNPLEWPERHVLQNLETFEKTKQKMRTGSLPHSSEQLLGHKEGPRDSITL
LDAKELLKYFTSDGLPIGDLQPLPIQKGEKTFVLTPELSPGKLQVLPFEK
ASVCHYHGIEYCLDDRKALERDGGFSELQSRLIRYETQTTCTRESFPVPT
VLSPLPSPVVSSDFGSVPDGEVLQNELRTEVSRLKRRSKDLNCLYPRKRL
VKSESSESLLSQTTGNSNHYHHHVTSRKPQTERSLPVTCPLVPIPSCETP
KLATKTSSGQKSMHESKTSRQIKESRSQKHTRILKEVVTETLKKHSITET
HECFTACSQRLFEISKFYLKDLKTSRGLFEEMKKTANNNAVQVIDWVLEK
TSKK

The protein sequence as been deduced from the following nucleotide sequence (SEQ ID No. 2):—

| | |
|---|---|
| atggatcggt acctgctgct ggtgatctgg ggggaaggaa aattcccgtc ggcggccagt | 60 |
| agggaggcag aacatgggcc agaggtgtcg tcgggtgagg gtactgagaa tcagccggac | 120 |
| ttcacagcag caaatgttta tcacctcttg aaaagaagca ttagtgcttc aattaatcca | 180 |
| gaagatagta ctttccctgc ctgttcagtg ggaggtatac ctggttccaa gaagtggttc | 240 |
| tttgcagtgc aggcaatata tggattttat cagttttgta gttctgattg gcaagagata | 300 |
| cattttgata cagaaaaaga taaaattgaa gatgttcttc aaacgaatat cgaagaatgt | 360 |
| ttgggtgctg ttgagtgttt tgaagaagaa gacagtaata gcagggaatc attatccttg | 420 |
| gctgatctct atgaagaagc tgcagaaaat ttgcatcagc tgtcagacaa gcttcctgct | 480 |
| cctggtagag caatggtaga tataatactg ttgctttctg acaaagatcc tcctaaattg | 540 |
| aaagactatt tacctactgt aggagcatta aaacatttga gagaatggta ttcagcaaag | 600 |
| atcactatag caggaaatca ttgtgaaata aactgtcaga aaattgcaga atacctttct | 660 |
| gctaatgttg tatctttaga agatctcaga aatgttattg actcaaagga attatggagg | 720 |
| gggaaaatac agatatggga aagaaagttt ggatttgaaa ttagttttcc tgaattttgt | 780 |
| ttaaagggag tcacacttaa gaattttagt acttctaatt taaatactga cttccttgcc | 840 |
| aaaaagatca taccatcaaa ggataagaat attttgccaa aggttttcca ttattatggc | 900 |
| cctgctttag aatttgtgca gatgataaaa ttatcagatc taccctcctg ctatatgtcg | 960 |
| gatattgaat ttgagttagg attgacaaac agtaccaaac agaattctgt gttgctgttg | 1020 |
| gagcagattt cttctctgtg tagcaaggtt ggtgctcttt ttgtattgcc atgtaccatt | 1080 |
| agtaacatac tgattccacc tcccaaccaa ctcagttcaa gaaaatggaa ggaatatata | 1140 |
| gctaaaaagc ctaaaacaat cagtgttcca gatgttgaag tgaaaggaga gtgttctagc | 1200 |
| tattatctct tgttacaagg taatggcaat agaagatgta aagccacatt gattcactca | 1260 |
| gccaaccaga tcaatggctc atttgcactc aatttaattc atggaaagat gaaaacaaag | 1320 |
| acagaagaag ccaaattgag ttttcctttt gacttattat cacttccaca tttttctggg | 1380 |
| gagcagattg tacagagaga gaaacagtta gctaatgttc aagttttagc tttggaagaa | 1440 |
| tgcctaaaaa gacgaaagtt ggcaaagcag cctgaaacag tttctgttgc tgaactcaaa | 1500 |
| agtctgttag tactcacaag gaaacacttt ttagattatt ttgatgctgt gattcctaaa | 1560 |
| atgattctaa gaaagatgga caaaattaaa accttcaata tattaaatga ttttagtcca | 1620 |
| gtggaaccta attcctcaag tctaatggaa accaatcctc tggaatggcc agaaaggcat | 1680 |
| gttcttcaaa atttggaaac ttttgaaaaa actaaacaaa aaatgagaac tggttcatta | 1740 |
| cctcattcat ctgaacagtt gctgggccac aaagagggtc ctcgggactc aatcacattg | 1800 |
| ttggatgcta aagaattgct gaagtacttt acctcagatg gattacccat tggagatctt | 1860 |
| caacctttac cgattcaaaa gggggaaaag acttttgttt tgacaccaga acttagtcct | 1920 |
| gggaaacttc aggtcttacc ttttgagaaa gcctcagtat gtcattatca tggaattgaa | 1980 |
| tattgcttgg atgaccgaaa agctttggaa agagatggag gattttctga acttcagtct | 2040 |
| cgtcttattc gttatgaaac tcaaactacc tgcaccagag aaagttttcc agtacctact | 2100 |
| gtgttgagcc ctcttccatc tcctgtagtt tcgtcagatc ctggaagtgt ccctgacgga | 2160 |
| gaagttttac aaaatgaact tcgaactgaa gtatcccgat tgaaacggag atctaaagat | 2220 |
| ctgaattgcc tttatcccag aaaaagactt gtgaaatctg aaagttcaga gtctcttctt | 2280 |
| tctcagacaa ctggtaatag taatcactat catcatcatg tgacatccag aaagccacaa | 2340 |
| acagaacggt ccttaccagt gacttgtcca ttggttccaa ttcctagctg tgaaactcca | 2400 |
| aaacttgcta caaagaccag ttcaggtcaa aaaagtatgc atgaatcaaa aacatcaagg | 2460 |
| caaattaagg aatcaagatc acagaaacac acacggatac tgaaagaagt agttactgaa | 2520 |
| accctgaaga aacacagtat taccgagact catgaatgtt tcactgcatg cagccagcgt | 2580 |
| ctctttgaaa tctctaagtt ctatctaaag gatcttaaaa cttcaagggg tctatttgaa | 2640 |
| gaaatgaaga aaacagcaaa caacaatgct gtacaggtga ttgactgggt attagaaaag | 2700 |
| acaagcaaga aatga | 2715 |

Reference to MTBP in this specification is preferably to the protein identified as SWISS-PROT Acc. No. Swiss-Prot/TrEMBL Q96DY7, and to functional variants thereof.

The inventors have found to their surprise that inhibition of MTBP activity is actually required for the effective treatment of cancer. This is the inverse to what may be expected from the prior art that suggested MTBP was tumour suppressive.

Although the inventors do not wish to be bound by any hypothesis, the inventors realised that MTBP has oncogenic effects when they analysed the data arising from the scientific investigations reported in Example 1. This data shows that MTBP increases ubiquitination and degradation of p53, whilst reducing auto-ubiquitination and thereby stabilising MDM2. Thus MTBP has the ability to differentially regulate the ubiquitin ligase activity of MDM2 towards itself and p53. The inventors believe this may explain the surprising anti-cancer effects that they have shown to be possessed by inhibitors according to the invention.

p53 and MDM2 are implicated in almost all human cancers and MTBP is expressed in a wide range of tissues at varying levels. It will therefore be appreciated that inhibitors according to the present invention are useful for treating a variety of cancer conditions. Thus, for example, the inhibitors may be used to treat leukaemia or cancer of the breast, oesphagus, stomach, pancreas, liver, kidney, small intestine, colon, uterus, ovaries, prostate, bladder, cervix, testes, brain or lungs. It is preferred that the inhibitors are used to treat cancers of the breast, lung and mesothelium.

Many types of cancer, especially carcinomas of the bladder, kidney, prostate and head & neck exhibit high levels of MDM2 and this is correlated with poor prognosis in a p53 status independent manner. The inventors have found that inhibition of MTBP reduces this effect of MDM2 in tumour cells whilst not leading to excessive apoptosis in normal cells. Accordingly it is preferred that medicaments according to the invention are used to treat such cancers

The inhibitors may also be used to prevent the development of cancer. For instance, the inhibitors may be given to subjects who are at risk (e.g. a genetic predisposition or adverse environmental exposure) of developing cancer. The inhibitors may also be used after surgery, radiotherapy or chemotherapy to prevent cancer re-establishing itself in a subject.

Inhibitors capable of decreasing the biological activity of MTBP may achieve their effect by a number of means. For instance, such inhibitors may:

(a) reduce interaction between MTBP and MDM2;

(b) compete with endogenous MTBP for MDM2 binding;

(c) bind to MTBP to reduce its biological activity; or

(d) decrease the expression of MTBP.

In a preferred first embodiment of the invention the inhibitor may directly interact with MTBP (e.g. (a)-(c) above).

Preferred inhibitors for use according to the first aspect of the invention comprise small molecule inhibitors. Such inhibitors may be identified as part of a high throughput screen of small molecule libraries. The screening method according to the sixth aspect of the invention (see below) represents a suitable means of identifying such inhibitors.

A preferred inhibitor according to this first embodiment is a neutralising antibody raised against MTBP. Such antibodies represent an important feature of the invention Thus, according to a fourth aspect of the invention, there is provided an antibody, or a functional derivative thereof, against MDM2 Binding Protein (MTBP).

The antibody preferably blocks MTBP interaction with MDM2. This may be by blocking the binding site on either protein.

Antibodies according to the invention may be produced as polyclonal sera by injecting antigen into animals. Preferred polyclonal antibodies may be raised by inoculating an animal (e.g. a rabbit) with antigen (e.g. MTBP or fragments thereof) using techniques known to the art.

Polyclonal antibodies, for use in treating human subjects, may be raised against a number of peptides derived from human MTBP (see Sequence above). For instance antibodies may be raised against PKTISVPDVEVKGEC (SEQ ID No. 3), RCKATLIHSANQING (SEQ ID No. 4), and TTCTRESFPVPT (SEQ ID No. 5).

A preferred polyclonal antibody is raised against the peptide CSSDWQEIHFDTE (SEQ ID No. 6) that lies between residues 93 and 106 inclusive of the human MTBP protein. This polyclonal antibody was raised using conventional techniques and is discussed in more detail in Example 2. CSSDWQEIHFDTE (SEQ ID No. 6) is preferred because this peptide is believed to be within the site on MTBP that is known to be involved in binding to MDM2.

Alternatively the antibody may be monoclonal. Conventional hybridoma techniques may be used to raise the antibodies. The antigen used to generate monoclonal antibodies according to the present invention may be the whole MTBP protein or a fragment thereof. Preferred fragments for generating the antibodies may also be the peptides discussed above and particularly CSSDWQEIHFDTE (SEQ ID No.6).

It is preferred that the antibody is a γ-immunoglobulin (IgG).

It will be appreciated that the variable region of an antibody defines the specificity of the antibody and as such this region should be conserved in functional derivatives of the antibody according to the invention. The regions beyond the variable domains (C-domains) are relatively constant in sequence. It will be appreciated that the characterising feature of antibodies according to the invention is the V_{H} and V_{L} domains. It will be further appreciated that the precise nature of the C_{H} and C_{L} domains is not, on the whole, critical to the invention. In fact preferred antibodies according to the invention may have very different C_{H} and C_{L} domains.

The inventors have found that antibodies, or functional derivatives thereof, according to the fourth aspect of the invention have surprising efficacy for preventing the development of cancer.

An antibody derivative may have 75% sequence identity, more preferably 90% sequence identity and most preferably has at least 95% sequence identity to a monoclonal antibody or specific antibody in a polyclonal mix. It will be appreciated that most sequence variation may occur in the framework regions (FRs) whereas the sequence of the CDRs of the antibodies, and functional derivatives thereof, is most conserved.

A number of preferred antibodies have both Variable and Constant domains. However it will be appreciated that antibody fragments (e.g. scFV antibodies) are also encompassed by the invention that comprise essentially the Variable region of an antibody without any Constant region.

Antibodies generated in one species are known to have several drawbacks when used to treat a different species. For instance when rodent antibodies are used in humans they tend to have a short circulating half-life in serum and may be recognised as foreign proteins by the patient being treated. This leads to the development of an unwanted human anti-rodent antibody response. This is particularly troublesome when frequent administrations of the antibody are required as it can enhance the clearance thereof, block its therapeutic effect, and induce hypersensitivity reactions. Accordingly preferred antibodies (if of non-human source) for use in human therapy are humanised.

Monoclonal antibodies are generated by the hybridoma technique. This usually involves the generation of non-human mAbs. The technique enables rodent monoclonal antibodies to be produced with almost any specificity. Accordingly preferred embodiments of the invention may use such a technique to develop monoclonal antibodies against MTBP. Although such antibodies are useful, it will be appreciated that such antibodies are not ideal therapeutic agents in humans (as suggested above). Ideally, human monoclonal antibodies would be the preferred choice for therapeutic applications. However, the generation of human mAbs using conventional cell fusion techniques has not to date been very successful. The problem of humanisation may be at least partly addressed by engineering antibodies that use V region sequences from non-human (e.g. rodent) mAbs and C region (and ideally FRs from V region) sequences from human antibodies. The resulting ‘engineered’ mAbs are less immunogenic in humans than the rodent mAbs from which they were derived and so are better suited for clinical use.

Humanised antibodies may be chimaeric monoclonal antibodies, in which, using recombinant DNA technology, rodent immunoglobulin constant regions are replaced by the constant regions of human antibodies. The chimaeric H chain and L chain genes may then be cloned into expression vectors containing suitable regulatory elements and induced into mammalian cells in order to produce fully glycosylated antibodies. By choosing an appropriate human H chain C region gene for this process, the biological activity of the antibody may be pre-determined. Such chimaeric antibodies offer advantages over non-human monoclonal antibodies in that their ability to activate effector functions can be tailored for cancer therapy, and the anti-globulin response they induce is reduced.

Such chimaeric molecules are preferred inhibitors for treating cancer according to the present invention. RT-PCR may be used to isolate the V_{H} and V_{L} genes from preferred mAbs, cloned and used to construct a chimaeric version of the mAb possessing human domains.

Further humanisation of antibodies may involve CDR-grafting or reshaping of antibodies. Such antibodies are produced by transplanting the heavy and light chain CDRs of a rodent mAb (which form the antibody's antigen binding site) into the corresponding framework regions of a human antibody.

Another preferred inhibitor according to the first embodiment of the invention is an inactive peptide fragment of MTBP which will compete with endogenous MTBP and thereby reduce its activity. For instance, the inventors have generated truncation mutants of MTBP that do not bind to MDM2 and which inhibit the ability of MTBP to inhibit MDM2. Although we do not wish to be bound by any hypothesis, this might suggest that MTBP binds to MDM2 as a dimer or higher oligomer. Examples of truncated mutants of MTBP that inhibit MDM2 activity and are therefore useful for treating cancer include: truncated proteins possessing amino acids 1-163, 1-191, 1-349, 1-374 or 1-681 amino acids from the human MTBP protein.

In a second embodiment of the invention the inhibitor may prevent or reduce expression of MTBP (i.e. (d) above). It is preferred that the inhibitor according to this embodiment is a gene-silencing molecule.

By the term “gene silencing molecule” we mean any molecule that interferes with the expression of the MTBP gene. Such molecules include, but are not limited to, siRNA, ribozymes and antisense. The use of such molecules represent an important aspect of the invention. Therefore according to a fifth aspect of the present invention there is provided the use of MTBP gene silencing molecule in the manufacture of a medicament for the treatment or prevention of cancer.

Gene silencing molecules may be antisense molecules (antisense DNA or antisense RNA) or ribozyme molecules. Ribozymes and antisense molecules may be used to inhibit the transcription of the MTBP gene. Antisense molecules are oligonucleotides that bind in a sequence-specific manner to nucleic acids, such as DNA or RNA. When bound to mRNA that has a complimentary sequence, antisense RNA prevents translation of the mRNA. Triplex molecules refer to single antisense DNA strands that bind duplex DNA forming a colinear triplex molecule, thereby preventing transcription. Particularly useful antisense nucleotides and triplex molecules are ones that are complimentary to or bind the sense strand of DNA (or mRNA) that encodes MTBP.

The expression of ribozymes, which are enzymatic RNA molecules capable of catalysing the specific cleavage of RNA substrates, may also be used to block protein translation. The mechanism of ribozyme action involves sequence specific hybridisation of the ribozyme molecule to complementary target RNA, followed by endonucleolytic cleavage, e.g. hammerhead motif ribozymes.

It is preferred that the gene-silencing molecule is a short interfering nucleic acid (siNA). The siNA molecule may be double-stranded and therefore comprises a sense and an antisense strand. The siNA molecule may comprise an siDNA molecule or an siRNA molecule. However, it is preferred that the siNA molecule comprises an siRNA molecule. Hence, the siNA molecule according to the invention preferably down-regulates gene expression by RNA interference (RNAi).

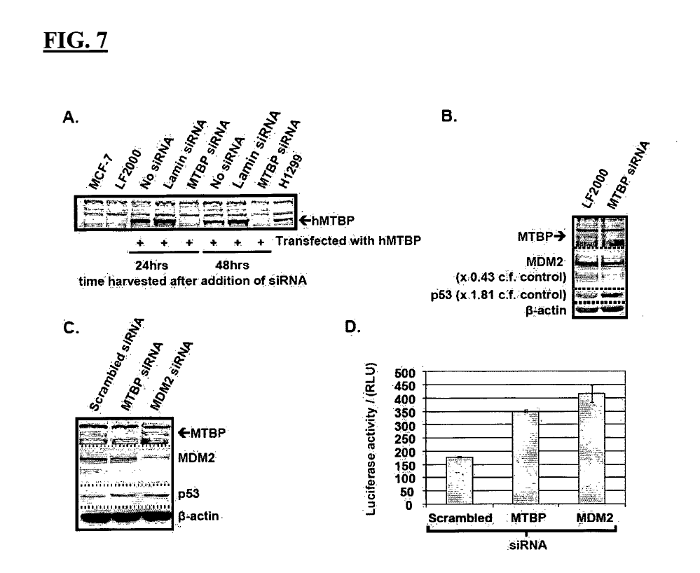

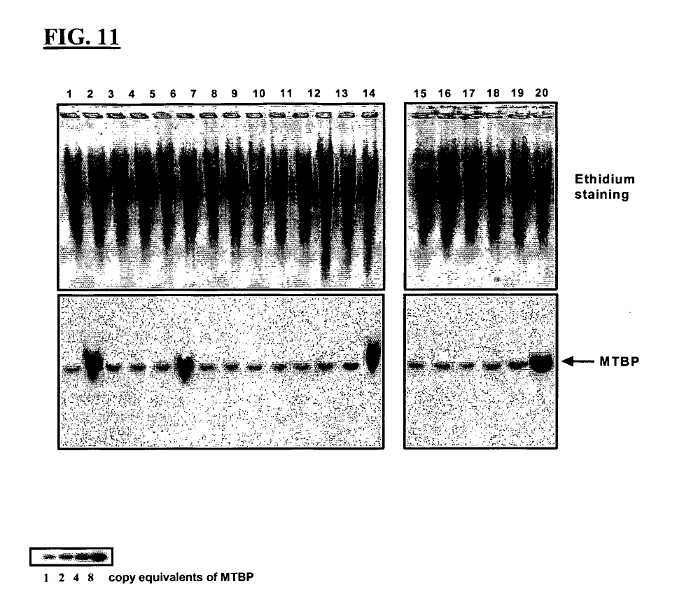

The inventors have demonstrated that the inhibition of MTBP in tumour cells, using RNAi, results in the inhibit of the growth of the cells (see Example 3). This data illustrates that RNAi may be used to provide a tumour specific growth inhibitory and/or apoptotic effect because they have found that:
i) MTBP expression is higher in many tumour cells (see FIGS. 11, 12 and 13 and table 3) and therefore the magnitude of reduction of MTBP will be greater (see FIG. 7).
ii) Recent studies demonstrate that p53 activity is not simply a function of the level of expression. Thus in tumour cells where p14^{ARF} is intact, p53 activation is determined by oncogenic stress and this leads to cell death and is the basis for the tumour suppressive effects of p53.

RNAi is the process of sequence specific post-transcriptional gene silencing in animals and plants. It uses small interfering RNA molecules (siRNA) that are double-stranded and homologous in sequence to the silenced (target) gene. Hence, sequence specific binding of the siRNA molecule with mRNAs produced by transcription of the target gene allows very specific targeted ‘knockdown’ of gene expression.

Preferably, the siNA molecule is substantially identical with at least a region of the coding sequence of the MTBP gene (see above) to enable down-regulation of the gene. Preferably, the degree of identity between the sequence of the siNA molecule and the targeted region of the MTBP gene is at least 60% sequence identity, preferably, at least 75% sequence identity, preferably at least 85% identity; preferably at least 90% identity; preferably at least 95% identity; preferably at least 97% identity; and most preferably, at least 99% identity.

Calculation of percentage identities between different amino acid/polypeptide/nucleic acid sequences may be carried out as follows. A multiple alignment is first generated by the ClustalX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof.

A substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention

Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequences which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine; large non-polar, hydrophobic amino acids include phenylalanine, tryptophan and tyrosine; the polar neutral amino acids include serine, threonine, cysteine, asparagine and glutamine; the positively charged (basic) amino acids include lysine, arginine and histidine; and the negatively charged (acidic) amino acids include aspartic acid and glutamic acid.

The accurate alignment of protein or DNA sequences is a complex process, which has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA.

Frequently, automatically generated alignments require manual alignment, exploiting the trained user's knowledge of the protein family being studied, e.g., biological knowledge of key conserved sites. One such alignment editor programs is Align (http://www.gwdg.de/˜dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable.

Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs.washington.edu/ phylip.html) using the “Similarity Table” option as the model for amino acid substitution (P). For DNA/RNA, an identical option exists within the DNADIST program of PHYLIP.

In a preferred embodiment, the inhibitor is an siNA molecule and comprises between approximately 5 bp and 50 bp, more preferably between 10 bp and 35 bp, even more preferably, between 15 bp and 30 bp, and yet still more preferably, between 16 bp and 25 bp. Most preferably, the siNA molecule comprises less than 22 bp.

Design of a suitable siNA molecule is a complicated process, and involves very carefully analysing the sequence of the target mRNA molecule. Then, using considerable inventive endeavour, the inventors have to choose a defined sequence of siRNA which has a certain composition of nucleotide bases, which would have the required affinity and also stability to cause the RNA interference.

The siNA molecule may be either synthesised de novo, or produced by a micro-organism. For example, the siNA molecule may be produced by bacteria, for example, E. coli.

Especially preferred siNA molecule sequences, which are adapted to down-regulate expression of the gene encoding MTBP comprise the following sequences:—

| | |
|---|---|
| 5′ GGCUCAUUUGCACUCAAUU 3′; | (SEQ ID No.7) |
| 5′ TCAAACGAATATCGAAGAA 3′; | (SEQ ID No.8) |
| 5′ AGATCCTCCTAAATTGAAA 3′; | (SEQ ID No.9) |
| 5′ AGAGTGTTCTAGCTATTAT 3′; | (SEQ ID No.10) |
| 5′ ACAGTTAGCTAATGTTCAA 3′; | (SEQ ID No.11) |
| 5′ ACAAAGATCCTCCTAAATT 3′; | (SEQ ID No.12) |
| 5′ AAAGATCCTCCTAAATTGA 3′; | (SEQ ID No.13) |
| 5′ CTTGGCTGATCTCTATGAA 3′; | (SEQ ID No.14) |
| 5′ GGAGAGTGTTCTAGCTATT 3′; | (SEQ ID No.15) |
| 5′ GTAGAGCAATGGTAGATAT 3′; | (SEQ ID No.16) |
| 5′ GCTATTATCTCTTGTTACA 3′; | (SEQ ID No.17) |
| 5′ TAGAGCAATGGTAGATATA 3′; | (SEQ ID No.18) |
| 5′ GAUCUACCCUCCUGCUAUAUU 3′; | (SEQ ID No.19) |
| and | |
| 5′ AAACGAAUAUCGAAGAAUGUU 3′. | (SEQ ID No.20) |

The siRNA of SEQ ID No. 5 is a most preferred siNA molecule for use according to the present invention.

It should be appreciated that such siNAs may comprise uracil (siRNA) or thymine (siDNA). Accordingly the nucleotides U and T, as referred to above, may be interchanged. However it is preferred that siRNA is used.

The inventors tested each of these siNA molecules by the methods as described in the Examples and demonstrated that these inhibitors were effective for reducing MTBP expression; reducing cell growth and are thereby effective for treating cancer.

Gene-silencing molecules used according to the invention are preferably nucleic acids (e.g. siRNA or antisense or ribozymes). Such molecules may (but not necessarily) be ones, which become incorporated in the DNA of cells of the subject being treated. Undifferentiated cells may be stably transformed with the gene-silencing molecule leading to the production of genetically modified daughter cells (in which case regulation of expression in the subject may be required, e.g. with specific transcription factors, or gene activators).

The gene-silencing molecule may be either synthesised de novo, and introduced in sufficient amounts to induce gene-silencing (e.g. by RNA interference) in the target cell. Alternatively, the molecule may be produced by a micro-organism, for example, E. coli, and then introduced in sufficient amounts to induce gene silencing in the target cell.

The molecule may be produced by a vector harbouring a nucleic acid that encodes the gene-silencing sequence. The vector may comprise elements capable of controlling and/or enhancing expression of the nucleic acid. The vector may be a recombinant vector. The vector may for example comprise plasmid, cosmid, phage, or virus DNA. In addition to, or instead of using the vector to synthesise the gene-silencing molecule, the vector may be used as a delivery system for transforming a target cell with the gene silencing sequence.

The recombinant vector may also include other functional elements. For instance, recombinant vectors can be designed such that the vector will autonomously replicate in the target cell. In this case, elements that induce nucleic acid replication may be required in the recombinant vector. Alternatively, the recombinant vector may be designed such that the vector and recombinant nucleic acid molecule integrates into the genome of a target cell. In this case nucleic acid sequences, which favour targeted integration (e.g. by homologous recombination) are desirable. Recombinant vectors may also have DNA coding for genes that may be used as selectable markers in the cloning process.

The recombinant vector may also comprise a promoter or regulator or enhancer to control expression of the nucleic acid as required. Tissue specific promoter/enhancer elements may be used to regulate expression of the nucleic acid in specific cell types, for example, mammory gland cells. The promoter may be constitutive or inducible.

Alternatively, the gene silencing molecule may be administered to a target cell or tissue in a subject with or without it being incorporated in a vector. For instance, the molecule may be incorporated within a liposome or virus particle (e.g. a retrovirus, herpes virus, pox virus, vaccina virus, adenovirus, lentovirus and the like). Alternatively a “naked” siNA or antisense molecule may be inserted into a subject's cells by a suitable means e.g. direct endocytotic uptake.

The gene silencing molecule may also be transferred to the cells of a subject to be treated by either transfection, infection, microinjection, cell fusion, protoplast fusion or ballistic bombardment. For example, transfer may be by: ballistic transfection with coated gold particles; liposomes containing an siNA molecule; viral vectors comprising a gene silencing sequence or means of providing direct nucleic acid uptake (e.g. endocytosis) by application of the gene silencing molecule directly.

In a preferred embodiment of the present invention siNA molecules may be delivered to a target cell (whether in a vector or “naked”) and may then rely upon the host cell to be replicated and thereby reach therapeutically effective levels. When this is the case the siNA is preferably incorporated in an expression cassette that will enable the siNA to be transcribed in the cell and then interfere with translation (by inducing destruction of the endogenous mRNA coding MTBP).

Inhibitors according to any embodiment of the present invention may be used in a monotherapy (e.g. use of siNAs or mAbs alone). However it will be appreciated that the inhibitors may be used as an adjunct, or in combination with, other cancer therapies (e.g. radiotherapy, conventional chemotherapy or even in conjunction with other oncogene gene silencing strategies). For instance, a combination therapy may comprise a gene silencing molecule according to the invention and a course of radiotherapy.

The inhibitors according to the invention may be contained within compositions having a number of different forms depending, in particular on the manner in which the composition is to be used. Thus, for example, the composition may be in the form of a capsule, liquid, ointment, cream, gel, hydrogel, aerosol, spray, micelle, transdermal patch, liposome or any other suitable form that may be administered to a person or animal suffering from cancer or at risk of developing a cancer. It will be appreciated that the vehicle of the composition of the invention should be one which is well tolerated by the subject to whom it is given, and preferably enables delivery of the inhibitor to the target site.

The inhibitors according to the invention may be used in a number of ways. For instance, systemic administration may be required in which case the compound may be contained within a composition that may, for example, be administered by injection into the blood stream. Injections may be intravenous (bolus or infusion), subcutaneous, intramuscular or a direct injection into the target tissue (e.g. an intraventricular injection—when used in the brain). The inhibitors may also be administered by inhalation (e.g. intranasally) or even orally (if appropriate).

The inhibitors may also be incorporated within a slow or delayed release device. Such devices may, for example, be inserted at the site of a tumour, and the molecule may be released over weeks or months. Such devices may be particularly advantageous when long term treatment with an inhibitor according to the invention is required and which would normally require frequent administration (e.g. at least daily injection).

It will be appreciated that the amount of an inhibitor that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the inhibitor within the subject being treated.

Optimal dosages to be administered may be determined by those skilled in the art, and will vary with the particular inhibitor in use, the strength of the preparation, the mode of administration, and the advancement or severity of the cancer. Additional factors depending on the particular subject being treated will result in a need to adjust dosages, including subject age, weight, gender, diet, and time of administration.

When the inhibitor is a nucleic acid conventional molecular biology techniques (vector transfer, liposome transfer, ballistic bombardment etc) may be used to deliver the inhibitor to the target tissue.

Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Generally, a daily dose of between 0.01 μg/kg of body weight and 0.5 g/kg of body weight of an inhibitor according to the invention may be used for the treatment of cancers, depending upon which specific inhibitor is used. When the inhibitor is an siNA molecule, the daily dose may be between 1 μg/kg of body weight and 100 mg/kg of body weight, and more preferably, between approximately 10 μg/kg and 10 mg/kg, and even more preferably, between about 50 μg/kg and 1 mg/kg.

When the inhibitor (e.g. antibody or siNA) is delivered to a cell, daily doses may be given as a single administration (e.g. a single daily injection). Typically, a therapeutically effective dosage should provide about 1 ng to 100 μg/kg of the inhibitor per single dose, and preferably, 2 ng to 50 ng per dose.

The inventors have found that providing siRNA every 2-3 days at a concentration of 20-40 nM at the target site is particularly effective. Accordingly such inhibitors do not have to be given on a daily basis but may be given approximately twice a week (e.g. a dose of approximately 150 μg/kg twice a week).

Antibody inhibitors may be administered in amounts between 10 μg/kg and 100 mg/kg; preferably in amounts between 100 μg/kg and 10 mg/kg; and more preferably may be administered at about. 1 mg/Kg. Such doses are particularly suitable when administered every few (e.g. every three) days.

Alternatively, some inhibitors, or cancer conditions, may require administration twice or more times during a day. As an example, siNA's according to the invention may be administered as two (or more depending upon the severity of the condition) daily doses of between 0.1 mg/kg and 10 mg/kg (i.e. assuming a body weight of 70 kg). A patient receiving treatment may take a first dose upon waking and then a second dose in the evening (if on a two dose regime) or at 3 or 4 hourly intervals thereafter. Alternatively, a slow release device may be used to provide optimal doses to a patient without the need to administer repeated doses.

Medicaments according to the invention should comprise a therapeutically effective amount of an inhibitor of MTBP activity and a pharmaceutically acceptable vehicle.

A “therapeutically effective amount” is any amount of an inhibitor according to the invention which, when administered to a subject inhibits cancer growth.

A “subject” may be a vertebrate, mammal, domestic animal or human being. It is preferred that the subject to be treated is human. When this is the case the inhibitors may be designed such that they are most suited for human therapy (e.g. humanisation of antibodies as discussed above). However it will also be appreciated that the inhibitors may also be used to treat other animals of veterinary interest (e.g. horses, dogs or cats).

A “pharmaceutically acceptable vehicle” as referred to herein is any physiological vehicle known to those of ordinary skill in the art useful in formulating pharmaceutical compositions.

In one embodiment, the medicament may comprise about 0.01 μg and 0.5 g of the inhibitor. More preferably, the amount of inhibitor in the composition is between 0.01 mg and 200 mg, and more preferably, between approximately 0.1 mg and 100 mg, and even more preferably, between about 1 mg and 10 mg. Most preferably, the composition comprises between approximately 2 mg and 5 mg of the inhibitor. The rest of the composition may comprise the velude.

Preferably, the medicament comprises approximately 0.1% (w/w) to 90% (w/w) of the inhibitor, and more preferably, 1% (w/w) to 10% (w/w). The rest of the composition may comprise the vehicle.

In a preferred embodiment, the pharmaceutical vehicle is a liquid and the pharmaceutical composition is in the form of a solution. In another embodiment, the pharmaceutical vehicle is a gel and the composition is in the form of a cream or the like.

Liquid pharmaceutical compositions which are sterile solutions or suspensions can be utilized by for example, intramuscular, intrathecal, epidural, intraperitoneal, intravenous, subcutaneous, intracerebral or intracerebroventricular injection. The inhibitor may be prepared as a sterile solid composition that may be dissolved or suspended at the time of administration using sterile water, saline, or other appropriate sterile injectable medium. Vehicles are intended to include, where appropriate, inert binders, suspending agents, lubricants, flavourants, sweeteners, preservatives, dyes, and coatings.

Knowledge of the surprising role MTBP plays in cancer has enabled the inventors to develop a screen for identifying whether or not test compounds are putative inhibitors for treating or preventing cancer. Thus, according to a sixth aspect of the present invention there is provided a method of screening a compound to test whether or not the compound has efficacy for treating or preventing cancer, comprising:
(i) exposing a biological system to the compound;
(ii) detecting the activity or expression of MTBP in the biological system; and;
(iii) comparing the activity or expression of MTBP in the biological system treated with the compound relative to activity or expression found in a control biological system that was not treated with the compound
wherein compounds with efficacy for treating or preventing cancer decrease activity or decrease expression of MTBP relative to the controls.

It will be appreciated that the method according to the sixth aspect of the invention may be adapted such that it is used to test whether or not a compound causes cancer. Therefore according to a seventh aspect of the invention there is provided a method of screening a compound, to test whether or not the compound causes cancer, comprising:
(i) exposing a biological system to the compound;
(ii) detecting the activity or expression of MTBP in the biological system; and
(iii) comparing the activity or expression of MTBP in the biological system treated with the compound relative to activity or expression found in a control biological system that was not treated with the compound
wherein compounds that are carcinogenic increase expression of MTBP relative to the controls.

The screening methods of the invention are based upon the inventors' realisation that the extent of MTBP expression and/or activity may be closely related to the development of cancer. The screening method of the sixth aspect of the invention is particularly useful for screening libraries of compounds to identify compounds that may be used as anti-cancer agents according to the first aspect of the invention. The seventh aspect of the invention may be used to identify compounds that are carcinogenic. Accordingly the screen according to the seventh aspect of the invention may be used for environmental monitoring (e.g. to test effluents from factories) or in toxicity testing (e.g. to test the safety of putative pharmaceuticals, cosmetics, foodstuffs and the like).

By “biological system” when mean any experimental system that would be understood by a skilled person to provide insight as to the effects a compound may have on MTBP activity or expression in the physiological environment. The system may comprise: (a) an experimental test subject when an in vivo test is to be employed; (b) a biological sample derived from a test subject (for instance: blood or a blood fraction (e.g. serum or plasma), lymph or a cell/biopsy sample); (c) a cell line model (e.g. a cell naturally expressing MTBP or a cell engineered to express MTBP); or even (d) an in vitro system that contains MTBP or its gene and simulates the physiological environment such that MTBP activity or expression can be measured.

The screen preferably assays biological cells or lysates thereof. When the screen involves the assay of cells, they may be contained within an experimental animal (e.g. a mouse or rat) when the method is an in vivo based test. Alternatively the cells may be in a tissue sample (for ex vivo based tests) or the cells may be grown in culture. It will be appreciated that such cells should express, or may be induced to express, functional MTBP. It is also possible to use cells that are not naturally predisposed to express MTBP provided that such cells are transformed with an expression vector. Such cells represent preferred test cells for use according to the sixth or seventh aspects of the invention. This is because animal cells or even prokaryotic cells may be transformed to express human MTBP and therefore represent a good cell model for testing the efficacy of candidate drugs for use in human therapy.

It is most preferred that biological cells used according to the screening methods of the present invention are derived from a subject and in particular xenograft models of cancer (e.g. mouse xenografts).

With regards to “detecting the activity or expression of MTBP” according to the screening methods of the present invention, by “activity” we mean the detection of MTBP—MDM2 binding or determination of an end-point physiological effect. By “expression” we mean detection of the MTBP protein in any compartment of the cell (e.g. in the cytosol, the Endoplasmatic Reticulum or the Golgi Apparatus); or detection of the mRNA encoding MTBP.

Expression of MTBP in the biological system may be detected by western blot, immuo-precipitation or immunohistochemistry.

The screening methods may also be based upon the use of cell extracts comprising MTBP. Such extracts are preferably derived from the cells described above.

The activity or expression of MTBP may be measured using a number of conventional techniques.

The test may be an immunoassay-based test. For instance, labelled antibodies (e.g. an antibody according to the fourth aspect of the invention with a conventional radiolabel or dye attached) may be used in an immunoassay to evaluate binding of a compound to MTBP in the sample. MTBP may be isolated and the amount of label bound to it detected. A reduction in bound label (relative to controls) would suggest that the test compound competes with the label for binding to MTBP and that it was also a putative anti-cancer agent.

Alternatively a functional activity measuring MTBP activity may be employed.

Furthermore molecular biology techniques may be used to detect MTBP in the screen. For instance, cDNA may be generated from mRNA extracted from tested cells or subjects and primers designed to amplify test sequences used in a quantitative Polymerase Chain Reaction to amplify from cDNA.

When a subject is used (e.g. an animal model or even an animal model engineered to express human MTBP), the test compound should be administered to the subject for a predetermined length of time and then a sample taken from the subject for assaying MTBP activity or expression. The sample may for instance be blood or biopsy tissue.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

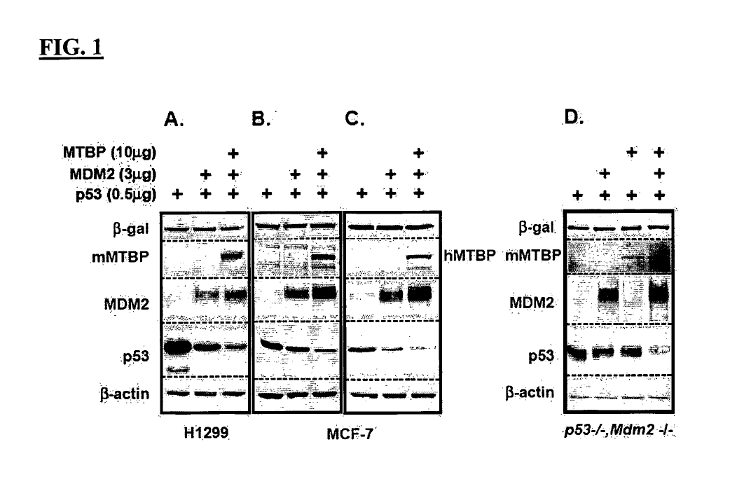

FIG. 1. Human (hMTBP) and Murine (mMTBP) MTBP promote stabilisation of MDM2 and consequent destabilisation of p53. Cells were transfected with the indicated amount of each plasmid. Total cell lysates were analysed by western blotting with the indicated antibodies. Note that in (a), (b) and (c) murine and human MTBP are detected with a polyclonal anti-MTBP serum: anti-sera#1 whilst in (d) the c-terminally HA-tagged mMTBP is detected with an anti-HA monoclonal antibody. Cells are (a) H1299, (b) and (c) MCF-7, and (d) Double-null (p53−/−, MDM2−/−) MEFs (mouse embryo fibroblasts).

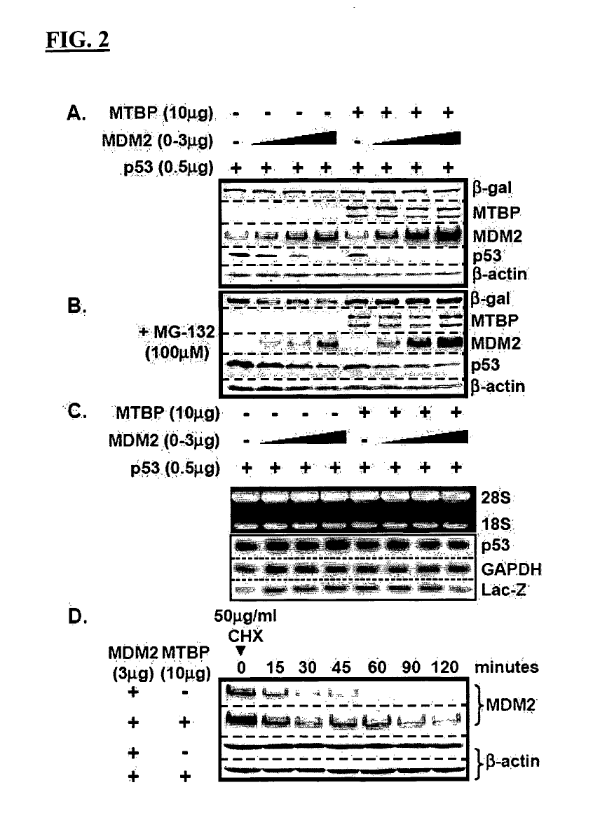

FIG. 2. MTBP promotes p53 degradation via a proteasome-dependent pathway. (a) H1299 cells were transfected with the indicated plasmids for 24 h. Three h prior to harvest, cells were treated with dimethylsulphoxide (DMSO). Cell lysates were then prepared and analysed by western blotting. (b) as (a), but cells were treated with the proteasome inhibitor, MG132 (100 μM), 3 h prior to harvest. (c) H1299 cells were transfected as in (a). After 24 h total cellular RNA was extracted and subjected to northern analysis using the indicated probes. The top panel shows an ethidium bromide-stained, agarose denaturing gel, loaded with 10 μg total cellular RNA from each transfection condition. (d) H1299 cells transfected as indicated were treated with 50 μg/ml cycloheximide and incubated for the times indicated. Cell lysates were then prepared and analysed by western blotting with the indicated antibodies. (e) MTBP promotes a reduction in p53 transcriptional activity. H1299 cells were transfected with the indicated plasmids for 24 h. Cells Were lysed and luciferase activity measured as described in materials and methods. Results are representative of three independent experiments. Data are shown as mean ±standard error of the mean. RLU, relative light units.

FIG. 3. Binding of MDM2 to p53 is necessary for MTBP to promote degradation of p53. (a) H1299 cells were transfected with the indicated plasmids for 24 h and cell lysates analysed by western blotting as indicated. (b) H1299 cells were transfected as indicated for 24 h. Six h prior to harvest, cells were subjected to 5Gy γ-irradiation and cell lysates analysed by western blotting as indicated.

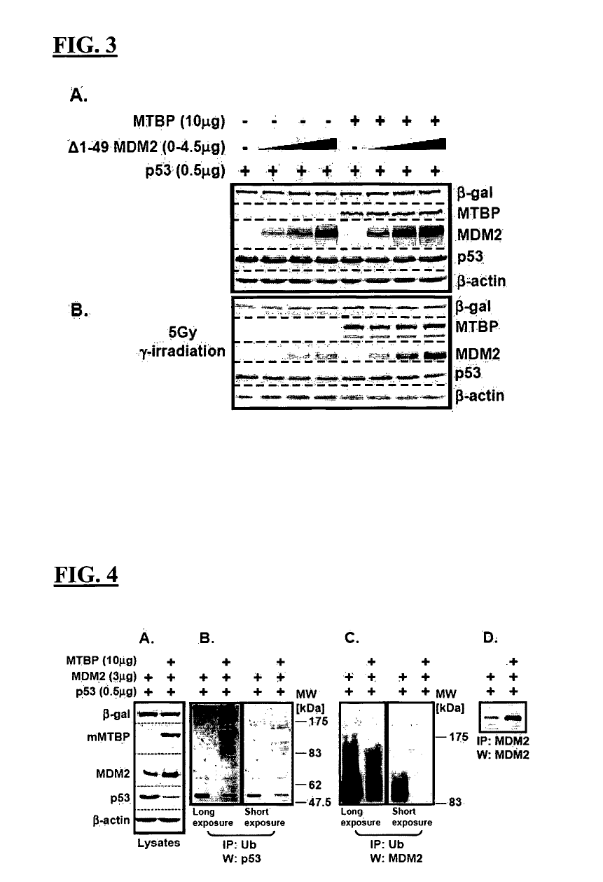

FIG. 4. MTBP induces an increase in the amount of ubiquitinated p53 and a decrease in the ubiquitination of MDM2. (a) H1299 cells were co-transfected as indicated. Total cell lysates were analysed by western blotting as indicated. (b) H1299 cells were co-transfected as in (a). Cellular extracts were immunoprecipitated with anti-ubiquitin P4D1 or isotype control antibody (not shown) followed by western analysis with an anti-p53 antibody (Ab2433). (c) H1299 cells were co-transfected as in (A), immunoprecipitated as in (b), and western blotted with an anti-MDM2 antibody (Ab-1). (d) Cell extracts prepared as for (b) and (c) were immunoprecipitated with anti-MDM2 SMP-14 followed by western blotting with an anti-MDM2 antibody (Ab-1).

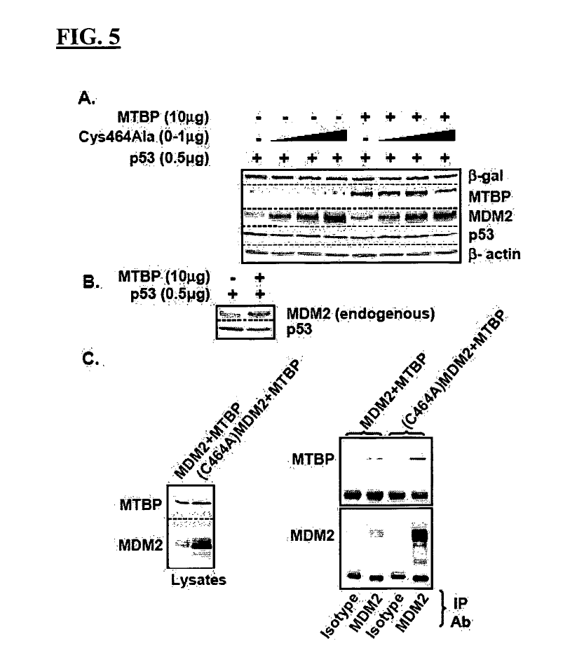

FIG. 5. The RING-finger domain of MDM2 is necessary for MTBP to promote degradation of p53. (a) H1299 cells were transfected with the indicated plasmids for 24 h and cell lysates analysed by western blotting. (b) Elements of Lanes 1 and 5 from (a) juxtaposed to facilitate visualisation of the effect of MTBP upon endogenous MDM2 and concomitantly upon transfected p53. In (c) and (d) H1299 cells were co-transfected with either wild type human MDM2 and MTBP (1:1 plasmid mass ratio) or with the RING-finger mutant of MDM2 (C464A) and MTBP (1:1 ratio) as indicated. (c) Shows the steady state protein levels in lysates from these cells analysed by western blotting as indicated. (d) Shows western blot analysis of immunoprecipitations performed on the same lysates. Cellular extracts were immunoprecipitated with either anti-MDM2 SMP14 antibody or an isotype control as indicated followed by immunoblotting with anti-HA antibody to detect MTBP or with anti-MDM2 antibody Ab-1 as indicated.

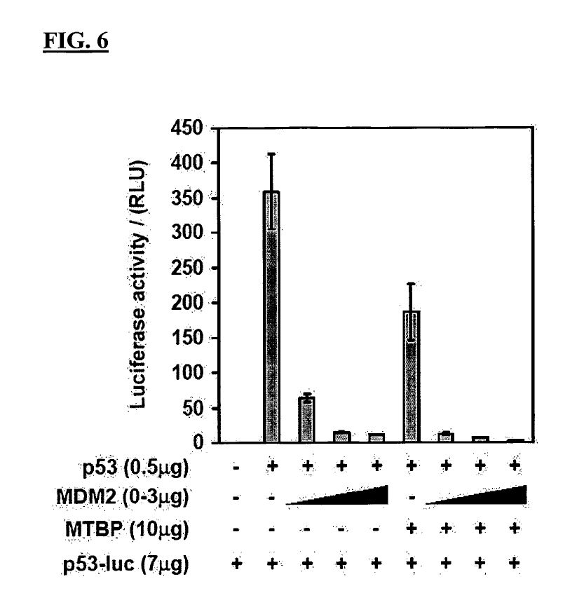

FIG. 6. MTBP inhibits MDM2 auto-ubiquitination directly in vitro.

In (a) and (b) 5 ng of MDM2 was incubated for the indicated times in the presence or absence of 5 μg of ubiquitin and 0 or 100 ng MTBP as shown. (a) Mono and poly-ubiquitin were detected with FK-2 and in (b) MDM2 was detected with Ab-1. Samples were resolved on a 6% acrylamide gel. (c) Colloidal coomassie stained 10% acrylamide gel of purified recombinant proteins used in (a) and (b).

FIG. 7. Endogenous MTBP regulates MDM2/p53 homeostasis. (a) MCF-7 cells were transfected with a plasmid that expresses human MTBP (hMTBP) as indicated and also with siRNA for the indicated times or treated with the transfection reagent alone (LF2000). MCF-7 and H1299 indicates un-treated cells. Lamin siRNA was used in this experiment as a negative control. Lysates from these cells were analysed by western blotting with an anti-MTBP serum as#1 to determine the steady state level of MTBP protein. (b) and (c) MCF-7 cells were transfected with the indicated siRNA and harvested 24 hours later. Lysates from these cells were analysed by western blotting as indicated. Densitometry in (b) was performed using KODAK ID version 3.5 software. (d) MCF-7 cells were transfected with the indicated siRNA and after 24 hours, cells were re-transfected with pp 53-TA-luc plasmid. Cells were lysed and luciferase activity measured as described in materials and methods. (e) Cells were transfected as in (d), harvested at 24 hours post transfection, fixed, stained with propidium iodide and analysed by FACS to determine the cell cycle profile. Results are representative of three independent experiments. Data are shown as mean ±standard error of the mean. RLU, relative light units.

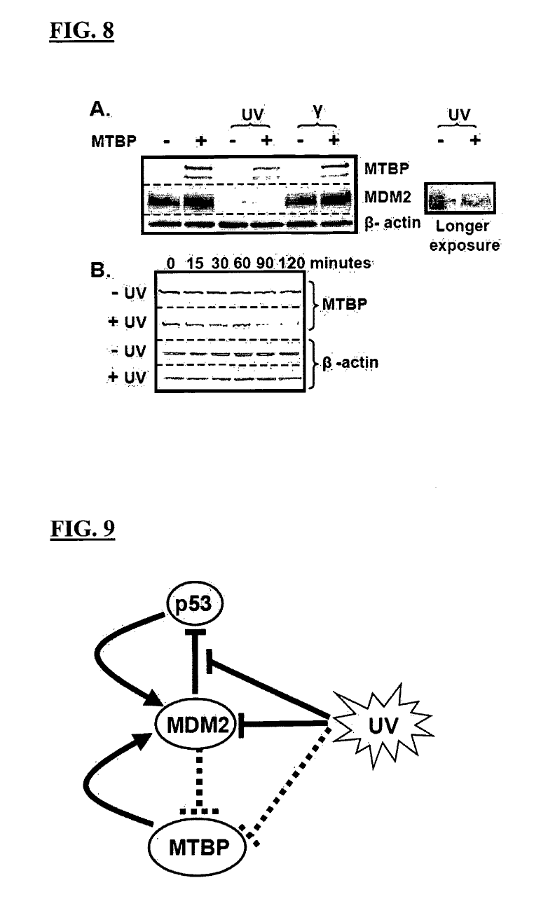

FIG. 8. MTBP stabilises endogenous MDM2 in unstressed cells and is destabilised following exposure of H1299 cells to UV-irradiation. (a) H1299 cells were transfected with MTBP (10 μg) or empty pCEP vector control for 24 h. Cells were either untreated prior to harvest or exposed to UV- (40 J/m²) or γ-irradiation (5Gy) 6 h prior to harvest. Cell lysates were then analysed by western blotting. On the right a longer exposure of the MDM2 track is shown from cells exposed to UV and transfected as indicated. (b) H1299 cells were transfected with MTBP (10 μg) for 24 h. Two h prior to harvest, cells were either left untreated or exposed to UV-irradiation (40 J/m²) and then all cells were treated immediately with an inhibitor of de novo protein synthesis (cycloheximide, 50 μg/ml). Cells were then harvested at the indicated times following addition of cycloheximide, and cell lysates analysed by western blotting as indicated.

FIG. 9. A proposed model of the relationship between MDM2, p53 and MTBP. Solid arrows are based upon experiments with physiological levels of MTBP and dashed arrows are based upon studies involving ectopic expression.

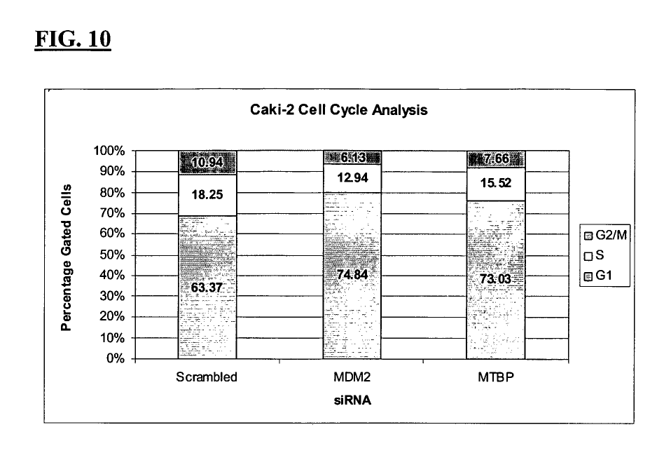

FIG. 10. Cell cycle analysis of cells transfected with the siRNAs of Example 3. Cells were fixed, stained with propidium iodide and analysed as previously described (Boyd et al. (2000) J Biol Chem 275:31883-90).

FIG. 11 Southern blot analysis of cancer cell lines and matched (from the same patient) normal EBV-transformed lymphoblastic lines for the MTBP gene. 10 μg of genomic DNA was digested with HindIII, eletrophoresed on a 0.7% agarose gel, transferred to Hybond XL and hybridised to a ³²P-labelled MTBP cDNA. Lanes are listed below:
1=normal CRL-2362
2=breast primary duct carcinoma CRL-2321
3=normal CRL-2337
4=breast primary duct carcinoma CRL-2336
5=normal CRL-5963
6=mesothelioma tumour CRL-5915
7=normal CRL-2339
8=breast ductal carcinoma CRL-2338
9=normal CRL-2346
10=breast primary duct carcinoma CRL-2314
11=normal CRL-5969
12=small cell lung carcinoma CRL-5929
13=normal CRL-2363
14=breast primary duct carcinoma CRL-2343
15=normal CRL-5961
16=lung adenocarcinoma CRL-5911
17=normal CRL-5967
18=non-small cell lung adenocarcinoma CRL-5985
19=normal CRL-5949
20=small cell lung carcinoma CRL-5858

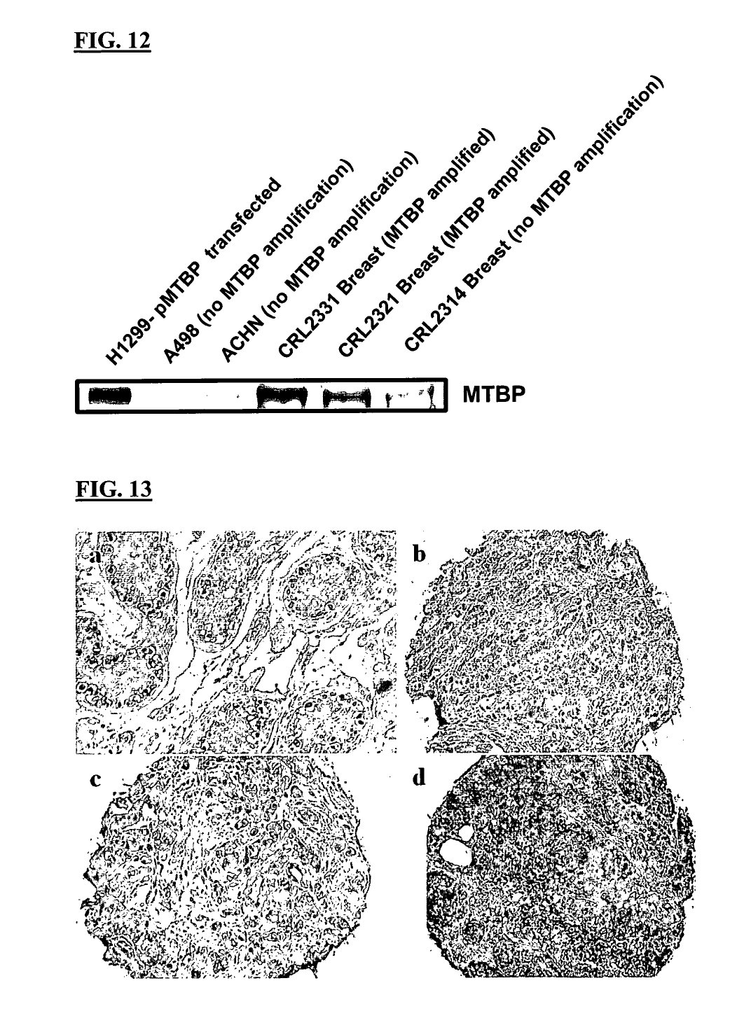

FIG. 12 Western blot analysis of total cell lysates from the indicated cell lines demonstrates that MTBP expression is relatively high in cell lines that harbour apparent amplification of the MTBP gene.

FIG. 13. IHC of breast cancer tissue with an anti-MTBP polyclonal. A total of 44 samples from a breast cancer tissue microarray were scored by a specialist breast pathologist. The figure shows illustrative examples of: a) normal breast, b) negative cancer, c) moderate cytoplasmic staining of cancer and d) strong cytoplasmic staining of cancer. Original images 100× magnification

### EXAMPLE 1

### 1.1 Introduction

Preliminary experiments were performed to evaluate the effect of MTBP on MDM2 and p53 activity. Prior art suggested that MTBP would be tumour suppressive. However the data reported here surprised the inventors and lead them to realise that inhibitors of MTBP activity were tumour suppressive.

The Example further illustrates that RNAi is effective for reducing MTBP expression and demonstrates that medicaments according to the invention may be used to treat cancers.

### 1.2 Methods

1.2.1 Plasmids and Antibodies

The p53 (pCEP4-hp53) and pMBP10 (pCEP4-mMTBP) expression constructs were described previously (Boyd et al. 2000 J Biol Chem 275:31883-90).

hMDM2:pCMVneobam was a kind gift from Dr. B. Vogelstein and is described in Oliner et al. (Nature 358: 80-83, 1992).

The human MDM2 RING-finger mutant (Cys464Ala):pCMVneobam3 was a kind gift of Dr. D. Xirodimas and is described in Xirodimas et al. (Oncogene, 20: 4972-4983, 2001).

The human Δ1-49 MDM2 clone was constructed from hMDM2:pCMVneobam by PCR and cloned into the Bam HI site of pCMVneobam using the following primers (supplied by MWG of Ebersberg, Germany) with flanking Bam HI restriction endonuclease sites and incorporating a Kozak consensus sequence:

(SEQ ID No.21)

5′-GAG AGG ATC CCC CGC CGC CCA CCA TGA AAG AGG TTC
TTT TTT ATC TTG G;
and

(SEQ ID No.22)

5′-GAG AGG ATC CCT AGG GGA AAT AAG TITA GCA CAA
TC.

The β-gal plasmid used as a transfection efficiency control has been described previously (Boyd et al. supra).

Human MTBP was cloned and sequenced from a human placental cDNA library and the construct for human MTBP expression was created by PCR with primers containing flanking Not I restriction endonuclease sites to amplify the full length ORF:

(SEQ ID No.23)

5′-GAG AGC GGC CGC ATC TCT GCG GCG ATG GAT CGG
TAC;
and

(SEQ ID No.24)

5′-GAG AGC GGC CGC TCA TTT CTT GCT TGT CTT TTC TAA
TAC.

Human MTBP was then sub-cloned into the Not I site of pCEP4 essentially as described elsewhere for the murine MTBP clone (Boyd, 2000 et al. supra).

pBlueBacHis2:MDM2 was created by sub-cloning a Bam HI fragment of MDM2 from hMDM2:pCMVneobam into the Bam HI site of pBlueBacHis2 (Invitrogen, USA).

pQE-p53 was created by subcloning a Bam HI/Xho I fragment of p53 from pCEP4-hp53 into the Bam HI/Sal I sites of pQE-31 (Qiagen, Germany).

pQE-hMTBP was created by subcloning a Sac I/Xho I fragment of hMTBP from pCEP4-hMTBP into the Sac I/Sal I sites of pQE-32 (Qiagen, Germany).

The following commercially available antibodies were used (the name in parenthesis being the manufacturers code for the antibody): Mouse monoclonal antibodies against human MDM2 (Ab-1 and Ab-2), p53 (Ab-6) and β-galactosidase (Ab-1) were purchased from Oncogene Research Products. The anti-actin antibody (C-2—used as a total protein loading control), anti-MDM2 SMP14 antibody and anti-ubiquitin P4D1 used for immunoprecipitation were purchased from Santa Cruz Biotechnology. Ab2433 rabbit polyclonal anti-p53 antiserum was obtained from Abcam. Leu™-16 antibody against CD20 used as an isotype control for immunoprecipitation was purchased from Becton Dickinson and the anti-haemagglutinin (HA) antibody used to detect HA-tagged MTBP (12CA5) was purchased from Roche Molecular Biochemicals. The anti-ubiquitin antibody FK2 which detects both mono- and poly-ubiquitinated proteins was purchased from Affiniti Research Products.

A rabbit polyclonal antibody (αs#1) was raised against a peptide fragment from human MTBP (CSSDWQEIHFDTE SEQ ID NO. 6) and this recognises both human (hMTBP) and murine MTBP (mMTBP) and represents a preferred inhibitor according to the first aspect of the invention.

1.2.2 Cell Culture and Transfection

H1299 (p53-null, human non-small cell lung carcinoma) and MCF-7 (mammary adenocarcinoma, ARF-null) cells were maintained in RPMI-1640 or DMEM medium respectively in the presence of 10% foetal calf serum with penicillin/streptomycin. MDM2/p53 double-null mouse embryo fibroblasts were maintained in high glucose DMEM medium in the presence of 10% foetal calf serum, 0.4% P-mercaptoethanol and penicillin/streptomycin. Sf9 and Hi5 insect cells were obtained from Invitrogen (Invitrogen, USA) and were maintained in a shaking incubator at 180 rpm and 28° C. in serum free SF-90011 or EX-CELL™ 405 media respectively.

Sf9 insect cells were co-transfected with pBlueBacHis2:MDM2 and Bac-N-Blue linear virus DNA™ using Cellfectin according to the manufacturer's instructions.

Mammalian cells were transiently transfected using 3 μl GeneJuice reagent (Novagan, UK) per microgram of DNA, and empty vector was used to ensure equal DNA content in transfections. In some experiments transfected cells were treated with the proteasome inhibitor, MG132 (100 μM) (Affiniti Research Products, UK) 3 hours prior to harvest, or with an inhibitor of de novo protein synthesis, cycloheximide (50 μg/ml)(VWR International, UK) 2 hours before harvesting.

siRNA was delivered to cells by transfection with Lipofectamine 2000 (Invitrogen, USA) according to the manufacturer's instructions.

The following siRNAs were used in this series of experiments:

(a) MTBP:

(SEQ ID No.7)

5′ GGCUCAUUUGCACUCAAUU 3′;
(b) a scrambled control for MTBP:

(SEQ ID No.25)

5′ GGACGCAUCCUUCUUAAUU 3′;
(c) MDM2:

(SEQ ID No.26)

5′ GCCACAAAUCUGAUAGUAU 3′;
and
(d) a Lamin control:

(SEQ ID No.27)

5′ CUGGACUUCCAGAAGAACA 3′

Each of these siRNas were synthesized by Dharmacon, USA.

In some experiments, cells were subjected to 5Gy gamma irradiation from a ¹³⁷CS source (Gammacell 1000, Atomic Energy of Canada Limited, now MDS Nordion) or 40 J/m² UV-irradiation from a 30 W UV lamp (Philips) calibrated using a Black-Ray® Model J-225 shortwave UV measuring meter (UVP, USA).

1.2.3 Western Analysis

Cells were harvested by trypsinisation after the indicated times and pelleted by centrifugation. Cell pellets were lysed in SLIP buffer (50 mM HEPES pH7.5, 10% glycerol, 0.1% Triton-X100, 150 mM NaCl) in the presence of the following protease inhibitors: aprotinin (2 μg/ml), leupeptin (0.5 μg/ml), pepstatin A (1 μg/ml), soybean trypsin inhibitor (100 μg/ml) and phenylmethylsulfonyl fluoride (PMSF) (1 mM). After 10 minutes incubation on ice, lysates were centrifuged at 20,000×g and protein concentrations in the supernatant were determined using Bradford reagent (BioRad). Fifty microgram samples of total protein in 1× protein sample buffer (50 mM Tris-HCl (pH 6.8), 2% SDS, 10% glycerol, 0.25% β-mercaptoethanol, bromophenol blue (1 mg/ml)) were separated by SDS-PAGE and transferred to Hybond ECL nitrocellulose membrane (Amersham Pharmacia Biotech, UK). Membranes were blocked in PBS-Tween-20 (0.1% v/v) containing non-fat dry milk (BioRad) (5% w/v) for 1 h at RT before incubation with primary antibodies (each at 3 μg/ml, except anti-p53 at 1 μg/ml and anti-MTBP at 1:1000). Membranes were washed 3 times for 15 minutes in PBS-Tween-20 before addition of HRP-conjugated anti-mouse (1:2500) or anti-rabbit (1:5000) secondary antibodies (Amersham Biosciences, UK) for 1 h at RT. Membranes were washed as before and signal was detected by Western Lightning Chemiluminescence Reagent (Perkin Elmer, USA).

1.2.4 Immunoprecipitation

Cells were harvested by trypsinisation and pelleted by centrifugation. Cell pellets were lysed in SLIP buffer plus BSA 0.5 mg/ml in the presence of aprotinin (2 μg/ml), leupeptin (0.5 μg/ml), pepstatin A (1 μg/ml), soybean trypsin inhibitor (100 μg/ml), phenylmethylsulfonyl fluoride (PMSF) (1 mM) and N-ethylmaleimide (NEM) (10 mM) for 10 minutes on ice. Cell lysates were then centrifuged at 20,000×g and protein concentration in the supernatant was determined using Bradford reagent (BioRad).

Four mg of cellular lysate was pre-cleared by incubating with Protein G Sepharose beads (Amersham Biosciences, UK) for 1 hr at 4° C. followed by brief centrifugation. Pre-cleared supernatants were then incubated with either 2 μg of anti-MDM2 SMP14 antibody or 2 μg of anti-Ubiquitin P4D1 antibody or with the same amount of the isotype control antibody Leu™-16 against CD20 for 1 hr at 4° C. Following this, the lysates were incubated with Protein G Sepharose beads for 2 hr at 4° C., the bead pellets were washed and re-suspended in 1× protein sample buffer prior to analysis by western blotting.

1.2.5 In Vivo Ubiquitination Assay

H1299 cells were co-transfected with either MDM2 and p53 (6:1 plasmid mass ratio) or with MTBP, MDM2 and p53 (20:6:1 ratio) expression plasmids using GeneJuice reagent as described above. Forty-eight hours after transfection cells were harvested. Samples were analysed by immunoprecipitation and/or western blotting as indicated.

1.2.6 Production and Purification of Recombinant Proteins

Recombinant MDM2 was produced in insect cells using the Bac-N-Blue system essentially as described by the manufacturer (Invitrogen, USA). Following transfection, MDM2 expressing plaques were identified in Sf9 cells, purified through three rounds of plaque purification and then virus stocks were produced using standard techniques. For MDM2 production, Hi5 cells were inoculated at multiplicity of infection of 1.0 with MDM2 baculovirus for 48 h. Cells were harvested by centrifugation and lysed in modified SLIP buffer: 300 mM NaCl, no BSA, 20 mM β-mercaptoethanol and protease inhibitors. The lysate was clarified at 12000 g for 15 minutes at 4° C. and then incubated for 90 minutes at 4° C. with Ni-NTA agarose (Qiagen, Germany). The beads were applied to a column and washed in modified SLIP buffer until the OD₂₈₀ was <0.01. MDM2 was eluted using a linear Imadazole gradient in modified SLIP buffer and MDM2 containing fractions were then dialysed overnight into ubiquitination assay buffer: Tris-HCl pH 7.6, 5 mM MgCl₂, 2 mM DTT, 20 μM ZnCl₂ and 10% glycerol. Recombinant p53 was expressed in XL-1 bacteria (Stratagene) from the construct pQE-p53 and purified under denaturing conditions by Ni⁺ affinity chromatography and FPLC using a Hi-Trap chelating column (Amersham). The column was washed with buffer (100 mM NaH₂PO₄, 10 mM Tris-HCL, 8M Urea) at pH 9, 6.3, 5.9 and then eluted in pH 4.1. Eluted protein was re-natured by overnight dialysis into ubiquitination assay buffer. Recombinant hMTBP was produced by the same method as recombinant p53 with the following modifications. Once protein was eluted into purification buffer pH 4.1, the pH was adjusted to 2.1 and the protein coupled to a SPFF Hi-Trap cation exchange column according to the manufacturers instructions (Amersham). Protein was then eluted with a linear 0-1M NaCl gradient. This was separated by SDS-PAGE and the full length protein was eluted from the gel using a model 422 elecro-eluter (Bio-Rad) according to the manufacturers instructions. Protein was then re-natured by overnight dialysis into ubiquitination assay buffer.

1.2.7 In Vitro Ubiquitination Assay

In vitro ubiquitination reactions were performed by incubating 5 mg ubiquitin (Boston Biochem) with 50 ng rabbit E1 (Boston Biochem), 200 ng E2 (UbCH5b) (Boston Biochem) and 5 ng E3 (baculovirus-expressed MDM2). Where indicated, bacterially expressed MTBP and/or p53 were included in the reaction. Assays were carried out in a 30 μl volume containing 50 mM Tris-HCl (pH 7.6), 5 mM MgCl₂, 2 mM ATP and 2 mM DTT. Following incubation at 37° C. reactions were stopped by addition of 10 μl 4× protein sample buffer and resolved on 6% SDS-polyacrylamide gels prior to analysis by western blotting

1.2.8 RNA Extraction and Northern Analysis

Total cellular RNA was extracted using RNA-Bee (Tel-Test,USA) according to the manufacturers instructions. Ten μg of total RNA was separated on a 1.2% agarose denaturing gel and transferred to Hybond ECL nitrocellulose membrane (Amersham Pharmacia Biotech, UK). Partial length probes for p53 (608 bp), glyceraldehyde 3-phosphate dehydrogenase (GAPDH) (233 bp) and lacZ (500 bp) were generated by PCR using p53:pCEP4, U2OS cell line cDNA and pFB-Neo-lacZ (Stratagene) respectively as templates. Primers used to generate fragments were:

p53:

(SEQ ID No.28)

5′-GGT TTC CGT CTG GGC TTC TT-3′;
and

(SEQ ID No.29)

5′-TTG GGC AGT GCT CGC TTA GT-3′.
GAPDH:

(SEQ ID No.30)

5′-TGC CGT CTA GAA AAA CCT GC-3′;
and

(SEQ ID No.31)

5′-ACC CTG TTG CTG TAG CCA AA-3′.
lacZ:

(SEQ ID No.32)

5′-CTC TGG CTC ACA GTA CGC GTA A-3′;
and

(SEQ ID No.33)

5′-CCA TCA ATC CGG TAG CTT TTC CG-3′.

Primers were supplied by MWG (Ebersberg, Germany). Probes were labelled with [α³²P]-dCTP using the Megaprime DNA labelling system (Amersham Pharmacia Biotech).

1.2.9 Luciferase Reporter Assay

For reporter assays cells were co-transfected with 7 μg per 10 cm dish (nominal) of a p53-responsive luciferase reporter construct pp 53-TA-luc (Mercury™ Pathway Profiling Systems, Clontech, USA).

Cells were lysed and luciferase activity measured 8 seconds after addition of sample to substrate using the Luciferase Assay Kit (Stratagene) with an integration period of 20 seconds in a TD 20/20 luminometer (Turner Design).

1.2.10 FACS Analysis

Cells were harvested and analysed by FACS essentially as described previously {Boyd, 2000 supra}. Cells were harvested 24 hours after addition of siRNA and washed in Dulbecco's phosphate buffered saline containing 1% bovine serum albumin (PB). Cells were then fixed in ethanol, stained in propidium iodide and analysed using a Beckman-Coulter EPICS cell sorter.

### 1.3 Results

The inventors originally identified MTBP as an MDM2 binding protein in a yeast two-hybrid screen and later confirmed the interaction in vitro and in vivo {Boyd et al. supra}. They were interested in the question: what is the consequence of MTBP binding upon MDM2 function? Although MDM2 has several effects upon p53 that contribute to its critical role as regulator of the “guardian of the genome”, one of the most important effects of MDM2 upon p53 is mediated through its ability to target p53 for degradation. They therefore investigated the effect of MTBP on the steady state levels of MDM2 and p53 in H1299 cells transfected with expression vectors for MTBP, MDM2 and p53 as indicated in FIG. 1a. As expected, addition of MDM2 resulted in a reduction in the steady state level of p53. However, the inventors were surprised to discover that addition of MTBP augmented this reduction, and moreover raised the steady state level of MDM2. They also investigated whether this effect occurred in other cell types and since the two most common cancers of the developed world are lung and breast cancer conducted studies in models of these cases (represented by H1299s: a non small cell lung carcinoma cell line and the breast adenocarcinoma cell line MCF-7). As shown in FIGS. 1b and 1c both murine (mMTBP) and human MTBP (hMTBP) promote an increase in the steady state level of MDM2 with a concomitant decrease in the level of p53 in MCF-7 cells. It is noteworthy that H1299s express p19^{ARF} whereas MCF-7 cells do not. Thus the effect that was observed is not dependent upon ARF status. To determine whether the effect of MTBP upon p53 was dependent upon MDM2, the inventors performed similar experiments in p53/Mdm2 null mouse embryo fibroblasts (double null MEFs). As shown in FIG. 1d, under conditions in which neither MDM2 (there being insufficient MDM2 to elicit a substantial effect) nor MTBP alone have any detectable effect upon p53 levels, addition of MTBP down-regulates p53 in an MDM2-dependent manner. Thus it was concluded that MDM2 is necessary for MTBP-enhanced down-regulation of p53.

The inventors found that titration of MDM2 was necessary to enable them to establish the sub-optimal conditions for MDM2 mediated degradation of p53, essential for the study of enhancement of MDM2 activity by MTBP. The inventors therefore developed a set of standard conditions in which p53 and MTBP input levels are constant but MDM2 is titrated as illustrated by FIG. 2a. By comparing lanes 1 and 5 it can be seen that even solely in the presence of endogenous MDM2, transfection of MTBP decreases the level of p53. This format also makes the effect of MTBP upon both MDM2 and p53 levels more apparent: compare lanes 2 and 6, 3 and 7 or 4 and 8. The inventors also examined these cells to determine whether MTBP alters the mRNA levels of p53 or MDM2 and have found that it does not, as illustrated in FIG. 2b.

Since the effect of MTBP upon p53 is dependent upon MDM2, the inventors then investigated whether this effect was mediated by proteasomal degradation. For these experiments cells were transfected as before and the proteasome inhibitor MG132 was added three hours before harvesting. FIG. 2c shows that addition of MG132 substantially rescues p53 from the effects of MDM2 (compare with FIG. 2a) in either the presence or absence of MTBP. It is important to appreciate that addition of MG132 occurs after MTBP has already increased the steady state level of MDM2. Thus, addition of MG132 would be expected to stabilise MDM2 to a higher level in the presence, compared to the absence, of MTBP (because there are more molecules of MDM2 at the time of adding MG132 in the presence of MTBP) and this is what was observed. The p53 profile shown in FIG. 2c clearly demonstrates that MDM2-mediated degradation of p53, in both the presence and absence of MTBP, can be substantially reversed by the addition of a proteasome inhibitor. If MTBP is increasing the steady state level of MDM2 in a proteasome dependent manner it was expected that MTBP would increase the half-life of MDM2. FIG. 2d shows that, in the presence of an inhibitor of de novo protein synthesis (cycloheximide), this is indeed the case. The inventors conclude that the effect of MTBP upon MDM2 and p53 is regulated at the level of protein turnover and this is substantially mediated by proteasomes. Having detected an effect of MTBP upon the steady state levels of p53, they then determined whether this was reflected by a reduction in p53 transcriptional activity. In FIG. 2e, the inventors measured the level of reporter gene expression from a p53-dependent luciferase construct. Addition of MTBP alone results in a 2-fold reduction in p53 transcriptional activity (compare lanes 2 and 6). It was concluded from a number of observations, including the data shown in FIG. 1d, that this is the result of MTBP stabilising endogenous MDM2 (as can also be seen most clearly in FIG. 5a, compare lanes 1 and 5 and emphasised in FIG. 5b) in these cells. Notwithstanding this, by comparing in FIG. 2e lanes 3 with 7 in particular (5.2 fold reduction with MTBP) but also 4 with 8 (2.2 fold) or 5 with 9 (4.3 fold), it can be seen that addition of MTBP leads not only to a reduction in the steady state protein level of p53 but also to a concomitant reduction in the level of p53 transcriptional activity in these cells.

Although the inventors thought it unlikely, they considered the possibility that the MDM2 dependent effect of MTBP upon p53 levels might not necessarily require binding of MDM2 to p53. They therefore created a mutant of human MDM2 lacking the first 49 amino acids that has been previously shown not to bind to p53, but which retains the ability to bind to MTBP {Boyd, supra}. As shown in FIG. 3a, Δ1-49 MDM2 has no effect upon the level of p53 in the presence or absence of MTBP. Note however that this mutant form of MDM2 is still stabilised by MTBP. To further investigate this question, the inventors next examined whether MTBP altered p53 steady state levels under conditions in which the interaction between MDM2 and p53 are blocked i.e. following exposure to ionising radiation. As illustrated in FIG. 3b they observed that p53 steady state levels were not affected by the addition of increasing amounts of MDM2, with or without transfection of MTBP, when cells were exposed to 5Gy of γ-irradiation. Interestingly, the inventors still observed an increase in MDM2 steady state levels under these conditions. MTBP retains the ability to bind to MDM2 under these conditions (not shown) and so it was concluded that the interaction between MTBP and MDM2 is not inhibited by ionising radiation. Since the inventors had consistently observed that MDM2 levels rose in the presence of MTBP and that the half-life of MDM2 is increased in the presence of raised levels of MTBP they wanted to examine whether this might be due to inhibition of MDM2 auto-ubiquitination. To examine this the inventors immunoprecipitated ubiquitinated proteins essentially as described (Bendjennat et al, 2003 all 114:599-610), in the presence and absence of transfected MTBP, and used western blot analysis to measure the electrophoretic patterns of MDM2 and p53 in the presence of an inhibitor of de-ubiquitination (N-ethylmaleimide). FIG. 4a shows the typical effect of MTBP expression upon MDM2 and p53 steady state levels in this experiment. It is striking that even though there is considerably less p53 present in MTBP transfected cells, when ubiquitinated proteins are immunoprecipitated (FIG. 4b), there is an increase in the level of slower migrating forms of p53 protein (ubiquitinated) in the presence of MTBP. The inventors therefore conclude that MTBP increases the ubiquitination of p53 and in so doing promotes p53 degradation. They have also examined the effect of MTBP upon MDM2 ubiquitination and as shown in FIG. 4c this is dramatically reduced in the presence of MTBP. Moreover, comparing FIGS. 4a, c and d, it is clear that the normal primary electrophoretic form of MDM2 (c.90 kDa) is markedly reduced and possibly even absent from the ubiquitin immunoprecipitation in the presence of MTBP, but is substantially increased in both the lysate and when an anti-MDM2 antibody is used for the immunoprecipitation. Thus in the presence of MTBP, this primary form of MDM2 is not bound to ubiquitin and ubiquitination of MDM2 is greatly reduced.

If MTBP stabilises MDM2 by inhibiting the auto-ubiquitination reaction, then a mutant of MDM2 that lacks ubiquitin ligase activity should be neither stabilised by MTBP nor should it display MTBP-mediated enhancement of p53 down-regulation. FIG. 5a shows that in H1299 cells the RING-finger mutant (Cys464Ala) of MDM2 is neither stabilised, nor stimulated to degrade p53 by the addition of MTBP, in spite of the fact that this mutant still binds to MTBP (see FIG. 5 panels c and d). It was concluded that MTBP acts to stabilise MDM2 by inhibiting the auto-ubiquitination reaction without inhibiting the ability of MDM2 to act as an E3 ligase for p53. It was further concluded that the ability of MTBP to stimulate MDM2-mediated down-regulation of p53 depends upon the ubiquitin ligase activity of MDM2 encoded by the RING-finger domain.

The next question asked was whether this effect of MTBP upon MDM2 was the result of direct interaction or whether it required additional factors. To address this, the inventors purified recombinant proteins and established an in vitro assay for MDM2 ubiquitination. Recombinant MDM2 purified from insect cells was mixed with recombinant MTBP purified from E. coli. As shown in FIG. 6, baculovirus expressed MDM2 is very rapidly (forms of MDM2 that do not enter the stacking gel are detectable within 1 minute (not shown)) auto-ubiquitinated. Nevertheless MDM2 auto-ubiquitination is very efficiently inhibited by addition of MTBP at a 10:1 molar ratio of MTBP:MDM2. Thus, MTBP is sufficient to directly inhibit MDM2 auto-ubiquitination in vitro.

To examine the physiological contribution of MTBP to MDM2 activity the inventors used siRNA. As shown in FIG. 7a, siRNA for MTBP down regulates transfected MTBP. This demonstrates that even supra-physiological levels of MTBP are effectively ablated by this siRNA. Using anti-MTBP serum (as#1) the inventors were able to detect, albeit weakly, endogenous MTBP in a range of cells. Note that the identity of the specific MTBP band has been confirmed in multiple systems including siRNA, peptide competition, MALDI-MS and transfection experiments (not shown).

FIG. 7b shows that the endogenous MTBP signal in MCF-7 cells is abolished by siRNA for MTBP. Under the conditions used, siRNA for MTBP also induces a significant reduction (2.3 fold) in endogenous MDM2 with a concomitant increase in the steady state level of endogenous p53 (1.8 fold). This effect on p53 steady state levels is comparable to the effect of transfecting siRNA for MDM2 as shown in FIG. 7c. This reduction is reflected in the level of p53 activity detectable in these cells. Aqs shown in FIG. 7d, siRNA for MTBP also induces a 2 fold increase in p53 transcriptional activity. This is comparable to the 2.3 fold increase in p53 transcriptional activity elicited by transfecting siRNA for MDM2. One of the consequences of increased p53 activity in these cells is a reduction in the growth rate and this can be seen in a transient transfection by an increase in the G₁ percentage of the population with associated reductions in S and G₂/M. The inventors therefore examined what happened when siRNA for MTBP or MDM2 were used to down-regulate these genes. As shown in FIG. 7e, the effect of down-regulation of MTBP is comparable to that observed when MDM2 is down-regulated by siRNA. In both cases there is a significant increase in the G₁ percentage with reductions in both S and G₂/M percentages. Thus, it was concluded that under normal growth conditions MTBP significantly contributes to MDM2/p53 homeostasis in these cells and through this to cell proliferation.

In unstressed cells MTBP is a co-factor for MDM2-mediated regulation of p53. What happens when cells are exposed to p53 activating stresses? The inventors had already seen that γ-irradiation resulted in MTBP-mediated stabilisation of MDM2 in the absence of any effect upon p53 (see FIG. 3b). There is an interesting difference between the regulation of the steady state level of MDM2 following exposure to ionizing and UV radiation. Following ionizing irradiation, MDM2 levels remain constant whereas after exposure to UV MDM2 is down-regulated. The inventors therefore examined the effect of UV-irradiation upon MTBP. Exposure of cells to 40 Jm⁻² UV-irradiation led to a reduction in the steady state level of the MTBP protein (as shown in FIG. 8a). This is due to a reduction in the half-life of the protein from >2 hours to c.60 minutes as FIG. 8b shows. It was concluded that MTBP augments the ubiquitin ligase activity of MDM2 in unstressed cells, but that in response to UV- (but not γ) irradiation, both MTBP and MDM2 destabilised as part of the cellular response to UV-induced DNA damage.

### 1.4 Discussion

These results show that the MDM2 binding protein MTBP alters the E3 ubiquitin ligase activity of MDM2 in vivo, such that it is stimulated with respect to p53 but inhibited with respect to MDM2. In support of this the inventors have shown that in transient transfection experiments both human and murine MTBP have a similar effect and this occurs in tumour cell lines of different origin (lung; H1299 and breast; MCF-7), and also in immortalised mouse embryo fibroblasts. The effect of ectopic expression of MTBP is entirely dependent upon the presence of MDM2 and is independent of the status of a known inhibitor of MDM2 ubiquitin ligase activity, p19^{ARF}, since H1299 cells possess wild-type ARF but MCF-7 cells have deletions of the ARF gene. This effect of MTBP depends upon MDM2 binding to p53 and is mediated by the RING domain of MDM2. In vitro MTBP is sufficient to inhibit MDM2 auto-ubiquitination. Using siRNA it was also shown that endogenous MTBP contributes to MDM2/p53 homeostasis in unstressed cells and thus to the down-regulation of p53 activity and cell cycle progression in cells. Finally, the inventors show that MTBP is destabilised as part of the cellular response to UV- but not γ-irradiation.

This data illustrated that inhibitors according to the invention are useful for suppressing growth of tumour cells and are therefore useful for treating cancer.

There are several issues raised by these experiments. In these studies with the proteasome inhibitor MG132 shown in FIG. 2c (the same effect was seen with MG115 and lactacystin), the inventors have seen that, although p53 is protected from degradation by MDM2 in both the presence and of course absence of MTBP, it is not possible to fully rescue p53 from degradation using proteasome inhibitors. MDM2 in contrast is very efficiently rescued using any one of a range of proteasome inhibitors and similar observations have been made by others. This may suggest that p53 degradation is not exclusively proteasomal or at least that, when proteasomes are inhibited, other pathways or proteolytic enzymes such as calpain compensate by inactivating p53. Presumably this is not the case for MDM2. Alternatively, the p53 in these cells may be partially stabilised as a result of low levels of cellular stress. The luciferase results are certainly compatible with this latter notion.

An important mechanistic question remains: how does MTBP increase ubiquitination of p53 whilst inhibiting MDM2 auto-ubiquitination? Either MTBP increases the kinetics of ubiquitin transfer by MDM2 or it stabilises MDM2 and thus increases the enzyme concentration with a concomitant increase in the reaction products (or both). In vitro as shown in FIG. 6, MTBP very effectively inhibits MDM2 auto-ubiquitination. Addition of MTBP also substantially reduces the level of ubiquitinated MDM2 in vivo (FIG. 4c). These observations suggest MTBP protects MDM2 from itself and by so doing increases the steady state concentration of MDM2 in the cell.

MTBP was named because of its MDM2 binding properties. Our results using an in vitro ubiquitination reaction clearly show that MTBP is sufficient to inhibit MDM2-auto-ubiquitination at a molar ratio of MTBP:MDM2 of 10:1. Even at a molar ratio of 3:1 there is substantial though incomplete inhibition (not shown). Thus direct interaction of MTBP with MDM2 or MDM2/E2-ubiquitin complexes are required for the effect of MTBP on MDM2 in vitro and therefore very likely in vivo. Based upon experiments such as those shown in FIG. 4 it is anticipated that MTBP prevents formation of covalent attachment of ubiquitin to MDM2 since there is a dramatic reduction in the amount of ubiquitin precipitable MDM2 when MTBP is transfected into these cells. The inventors do not yet know whether this occurs as a result of preventing interaction with the E2 although this would seem unlikely given the increased p53 ubiquitination that we have detected in vivo. Interestingly, they have also performed these in vitro ubiquitination experiments with c-terminally truncated forms of MTBP and have found these to have no effect upon MDM2 auto-ubiquitination, even when present at molar ratios in excess of 1000:1. Thus it is likely, if not surprising, that forms of MTBP that cannot bind to MDM2 also cannot inhibit MDM2 auto-ubiquitination.

To investigate the physiological relevance of the function of MTBP the inventors have examined the effect of ablation of endogenous MTBP using siRNA. As shown in FIG. 7 this results in a reduction in the steady state level of MDM2 with a concomitant approximately 2 fold increase in the p53 steady state level which results in a 2 fold increase in p53-dependant transcription. Given that the ablation of MDM2 with siRNA leads to an approximately 2.3-fold increase in p53 activity, and a similarly altered cell cycle profile the inventors have concluded that MTBP contributes in a significant way to the regulation of MDM2/p53 homeostasis in unstressed cells. A priori one would anticipate that MTBP might not have this effect under conditions of cellular stress and the inventors have seen, either directly through down-regulation by UV-irradiation or indirectly via γ-irradiation induced dissociation of MDM2 from p53 that this is indeed the case.

Taking all of these data together the inventors arrive at a model that integrates MTBP into the MDM2-p53 pathway as illustrated in FIG. 9. Similar to the interaction of p53 with MDM2, there may exist a feedback loop between MDM2 and MTBP. The prior art reports that high-level expression of MTBP induces growth arrest in a p53-independent manner. However, those data demonstrate that MDM2-mediated ubiquitination of p53 is stimulated by MTBP. It is possible that ectopic expression of MTBP leads to supra-physiological stimulation of MDM2 leading to the degradation of additional targets, though clearly this possibility requires investigation. Indeed, transfection of the MDM2 cDNA inhibits cell proliferation in many cell lines. A recent study found that over-expression of truncated forms of MDM2 could also inhibit proliferation in primary MEFs and that the RING-finger domain was both necessary and sufficient for this effect. Could supra-physiological MTBP induce growth arrest by altering MDM2 activity, similar to the effect of transfection of truncated forms of the gene? This possibility may be increasingly attractive given that MTBP has differential effects upon the cis and trans ubiquitination activities of MDM2. The inventors have not observed anything that would indicate that MTBP is targeted for degradation by MDM2. Therefore the ability of MDM2 to abrogate the growth inhibitory activity of MTBP most likely requires an alternative explanation. MTBP is a protein of c.900 amino acids in both human and mouse. There is little homology to any other known mammalian gene with the only identified similarity to the yeast BOI genes being located in the carboxy-terminus. This same region is necessary for interaction of MTBP with MDM2. It is quite possible therefore that other domains of MTBP mediate a growth inhibitory activity when expressed supra-physiologically. This effect would have to retain sensitivity to MDM2, and one way to envisage this dominance of MDM2 might be through regulation of MTBP sub-cellular distribution, as occurs with some other MDM2 interacting proteins. These studies to date have utilised different ratios of MTBP and MDM2 plasmids and that for MTBP stabilisation of MDM2, MTBP is in excess by plasmid weight whereas for MDM2 abrogation of MTBP mediated growth arrest the converse is true. Whatever the mechanism, we have identified several additional cell lines that are also sensitive to MTBP-induced growth arrest (data not shown) and clearly this effect of MTBP is a general one.

The p53-MDM2 autoregulatory pathway is becoming more and more complex. In addition to well studied molecules that impinge on the pathway such as ARF and MDMX, new members are still being discovered such as COP1 and Pirh2. We now add a previously described MDM2 binding protein MTBP to this mix in a previously unsuspected role as a co-factor for MDM2.

It is vitally important for a metazoan organism to tightly and precisely regulate the activity of p53 to prevent inappropriate activation leading to cellular, or more catastrophically, organismal death. To achieve this tight regulation it is necessary to maintain the correct balance between p53 and MDM2 in every tissue in the body and the levels of p53 and MDM2 vary considerably from tissue to tissue. MDM2 is constitutively expressed at a wide range of levels in many cell types with the highest levels of expression being found in the testis and ovaries. Interestingly, these same tissues also express the highest levels of MTBP mRNA and protein (unpublished results). Coincidentally, the substantially overlapping patterns of high expression of MTBP and MDM2 occur in tissues in which there are high levels of proliferation, and it is perhaps not surprising that these same tissues are (according to the current ICRP 1990) also the most radio-sensitive in the body. In these cells there may be a greater need to ensure that p53 is not inadvertently activated and by expressing MTBP these cells increase the effect of MDM2 and thus, presumably, reduce this risk. The difference in regulation of MDM2 and MTBP stability in response to exposure to UV and y radiation is intriguing. Clearly, this difference must reflect upstream differences in the response to these forms of cellular stress and it will be interesting to investigate further whether there is a connection between MTBP stability and activation of, for example, ATR by UV. MDM2/p53 homeostasis must be maintained for mammalian viability. By inhibiting the cis and promoting the trans reaction of the MDM2 E3 ubiquitin ligase, the role that we have discovered for MTBP may well provide a function that is essential for life.

### EXAMPLE 2

Having established that inhibitors of MTBP were effective for treating cancer, the inventors raised both polyclonal and monoclonal antibodies that were found to be useful for treating cancer according to the present invention.

2.1 Polyclonal Antibody to MTBP

A rabbit polyclonal anti-peptide antibody (designated αS#1) that recognises an epitope CSSDWQEIHFDTE (SEQ ID No. 6) that lies between residues 93 and 106 inclusive of the human MTBP protein was raised using conventional techniques.

This polyclonal antibody (Pab) recognises both human and murine MTBP in immunofluorescence, immunohistochemistry (IHC) and by western blot. In mouse, the epitope is largely conserved there being one semi-conservative substitution (underlined): CSSDWQEIHFDAE. (SEQ ID No. 34)

2.2 Monoclonal Antibody to MTBP

A monoclonal antibody for MTBP was made using conventional hybridoma technology.

The antibodies were raised against whole MTBP. Multi-milligram quantities of recombinant MTBP were produced in E. coli and purified to >95% purity. This was used for immunisation and hybridoma production. Most efficacious clones were selected.

### EXAMPLE 3

The inventors designed a number of siRNA molecules that may be used according to the invention. A specific siRNA molecule was based on the MTBP target sequence: 5′ GGCUCAUUUGCACUCAAUU 3′ (SEQ ID No. 7). This siRNA molecule was demonstrated to be effective for reducing the level of MTBP expressed in a range of cell types.

Transfection of 40 nM siRNA with lipofectamine 2000™ (Invitrogen) reduces the level of MTBP expression by c.80% or more depending upon the cell type. By reducing the level of MTBP cells arrest in the G₁ phase of the cell cycle to a similar degree to that observed with siRNA for MDM2 (see FIG. 10). This illustrates that siRNA according to the invention is effective for the treatment of cancer.

### EXAMPLE 4

p53 and MDM2 are implicated in almost all human cancers and MTBP is expressed in a wide range of tissues at varying levels. The inventors performed further experiments to demonstrate the correlation between MTBP expression and carcinogenesis. It will be appreciated that cancers demonstrating high levels of MTBP expression may be advantageously treated according to the invention.

Tests were conducted on cells from breast, lung and mesothelium and evidence was found of MTBP gene amplification in all three (as illustrated in FIG. 11 and summarised in table 1).

TABLE 1
Cell lines harbouring putative MTBP amplification

| Number | Type |
|---|---|
| CRL-2321 | Breast primary ductal carcinoma |
| CRL-2331 | Breast primary ductal carcinoma |
| CRL-2343 | Breast primary ductal carcinoma |
| CRL-2324 | Lung adenocarcinoma |
| CRL-5858 | Lung small cell carcinoma |
| CRL-5872 | Lung non-small cell adenocarcinoma |
| CRL-5922 | Lung non-small cell adenocarcinoma |
| CRL-5915 | Mesothelioma |

Essentially, it was found by Southern-blot analysis that the MTBP gene is over-represented (c.4 to >8-fold) in 8/20 tumour-derived lines but not matched lymphoblasts from the same patients (0/20). MTBP lies close to c-MYC, therefore MTBP may reside in c-MYC amplicons. However, whether or not MTBP is co-amplified with c-MYC, amplification of the MTBP gene resulting in over-expression of MTBP protein could stabilise MDM2 protein. In at least two cases examined to date the inventors found that MTBP amplification does lead to high levels of MTBP protein (FIG. 12).

This data illustrates that up-regulation of MTBP promotes cancer in almost any tissue. Accordingly inhibitors according to the invention, which inhibit MTBP, may be used to treat any cancer type.

### EXAMPLE 5

More detailed studies were performed to investigate the correlation between MTBP expression and the development of breast cancer, seminomas and bladder cancer

5.1 Breast Cancer

Samples were stained with an anti-MTBP antibody (see above) and were also scored by a specialist breast pathologist to investigate whether or not MTBP expression correlated with microscopic assessment of cancer development

FIG. 13 provides illustrative examples of Immunohistochemical (IHC) staining of breast cancer tissue with an anti-MTBP polyclonal. Variable staining for MTBP can be seen in: a) normal breast, b) negative cancer, c) moderate cytoplasmic staining of cancer and d) strong cytoplasmic staining of cancer. Original images 100× magnification.

In total 44 samples from a breast cancer tissue microarray were scored and examined by IHC with anti-MTBP antibody. Table 2 illustrates the total results for these experiments.

TABLE 2
Summary of results of IHC analysis of MTBP
expression in breast cancer.

| | | Number |
|---|---|---|
| Score | (description) | Observed |
| 0 | (negative) | 10 |
| 2c | (moderate cytoplasmic) | 27 |
| 2nc | (moderate nuclear and cytoplasmic) | 5 |
| 3c | (strong cytoplasmic) | 2 |

It can be seen from Table 2 that 76% of the cancers were positive for MTBP expression whereas no expression was detected in normal breast tissue (data not shown).

This illustrates that MTBP expression is associated with breast cancer.

Medicaments according to the invention may be used to reduce MTBP expression (e.g. as illustrated above for RNAi in Example 1) and will therefore be particularly useful for treating breast cancer. Accordingly a skilled person will appreciate that such medicaments may be used to treat breast cancer, and other cancers, by reducing MTBP activity.

5.2 Seminomas and Bladder Cancer

Similar experiments (data not presented) illustrated that MTBP expression is correlated with a number of other specific cancer. For instance, a correlation between MTBP expression and tumour development in seminomas was observed. MTBP staining is seen in the tumours cells of seminomas and embryonal carcinoma (as well as the precursor lesion—Intratubular Germ Cell Neoplasia ITGCN) whereas, in normal tubules, spermatogonia and spermatids appear to stain with a variable intensity. A similar correlation was established between the development of bladder cancer and MTBP expression.

1. A composition comprising an inhibitor of MDM2 Binding Protein (MTBP) activity.

2. The composition according to claim 1 wherein the inhibitor:
(a) reduces interaction between MTBP and MDM2;
(b) competes with endogenous MTBP for MDM2 binding;
(c) binds to MTBP to reduce its biological activity; or
(d) decreases the expression of MTBP.

3. The composition according to claim 1 wherein the inhibitor prevents or reduces expression of MTBP.

4. The composition according to claim 3 wherein the inhibitor is a gene-silencing molecule.

5. The composition according to claim 4 wherein the gene-silencing molecule is a ribozyme or an antisense molecule.

6. The composition according to claim 4 wherein the gene-silencing molecule is a short interfering nucleic acid (siNA).

7. The composition according to claim 6 wherein the siNA is siRNA.

8. The composition according to claim 6 wherein the siNA is one of:

| | |
|---|---|
| 5′ GGCUCAUUUGCACUCAAUU 3′; | (SEQ ID No.7) |
| 5′ TCAAACGAATATCGAAGAA 3′; | (SEQ ID No.8) |
| 5′ AGATCCTCCTAAATTGAAA 3′; | (SEQ ID No.9) |
| 5′ AGAGTGTTCTAGCTATTAT 3′; | (SEQ ID No.10) |
| 5′ ACAGTTAGCTAATGTTCAA 3′; | (SEQ ID No.11) |
| 5′ ACAAAGATCCTCCTAAATT 3′; | (SEQ ID No.12) |
| 5′ AAAGATCCTCCTAAATTGA 3′; | (SEQ ID No.13) |
| 5′ CTTGGCTGATCTCTATGAA 3′; | (SEQ ID No.14) |
| 5′ GGAGAGTGTTCTAGCTATT 3′; | (SEQ ID No.15) |
| 5′ GTAGAGCAATGGTAGATAT 3′; | (SEQ ID No.16) |
| 5′ GCTATTATCTCTTGTTACA 3′; | (SEQ ID No.17) |
| 5′ TAGAGCAATGGTAGATATA 3′; | (SEQ ID No.18) |
| 5′ GAUCUACCCUCCUGCUAUAUU 3′; | (SEQ ID No.19) |
| or | |
| 5′ AAACGAAUAUCGAAGAAUGUU 3′. | (SEQ ID No.20) |

9. The composition according to claim 1 wherein the inhibitor is a neutralising antibody raised against MTBP or a fragment thereof.

10. The composition according to claim 9 wherein the antibody is a polyclonal antibody.

11. The composition according to claim 9 wherein the antibody is a monoclonal antibody.

12. The composition according to claim 9 wherein the antibody is raised against the peptide CSSDWQEIHFDTE (SEQ ID No. 6).

13. The composition according to claim 9 wherein the antibody is raised against the whole of MTBP.

14. The composition according to claim 1 wherein the inhibitor is an inactive peptide fragment of MTBP that competes with endogenous MTBP and thereby reduces its activity.

15-18. (canceled)

19. A method of treating or preventing cancer comprising administering to a subject in need of such treatment a therapeutically effective amount of an inhibitor of MDM2 Binding Protein (MTBP) activity.

20. (canceled)

21. A method of screening a compound to test whether or not the compound has efficacy for treating or preventing cancer, comprising:
(i) exposing a biological system to the compound;
(ii) detecting the activity or expression of MTBP in the biological system; and;
(iii) comparing the activity or expression of MTBP in the biological system treated with the compound relative to activity or expression found in a control biological system that was not treated with the compound
wherein compounds with efficacy for treating or preventing cancer decrease activity or decrease expression of MTBP relative to the controls.

22. An anticancer agent identified according to the method of claim 21.

23. A method of screening a compound, to test whether or not the compound causes cancer, comprising:
(i) exposing a biological system to the compound;
(ii) detecting the activity or expression of MTBP in the biological system; and
(iii) comparing the activity or expression of MTBP in the biological system treated with the compound relative to activity or expression found in a control biological system that was not treated with the compound
wherein compounds that are carcinogenic increase expression of MTBP relative to the controls.

24. The method according to claim 19 wherein the inhibitor:
(a) reduces interaction between MTBP and MDM2;
(b) competes with endogenous MTBP for MDM2 binding;
(c) binds to MTBP to reduce its biological activity; or
(d) decreases the expression of MTBP.

25. The method according to claim 19 wherein the inhibitor prevents or reduces expression of MTBP.

26. The method according to claim 25 wherein the inhibitor is a gene-silencing molecule.

27. The method according to claim 26 wherein the gene-silencing molecule is a ribozyme or an antisense molecule.

28. The method according to claim 26 wherein the gene-silencing molecule is a short interfering nucleic acid (siNA).

29. The method according to claim 28 wherein the siNA is siRNA.

30. The method according to claim 28 wherein the siNA is one of:

| | |
|---|---|
| 5′ GGCUCAUUUGCACUCAAUU 3′; | (SEQ ID No.7) |
| 5′ TCAAACGAATATCGAAGAA 3′; | (SEQ ID No.8) |
| 5′ AGATCCTCCTAAATTGAAA 3′; | (SEQ ID No.9) |
| 5′ AGAGTGTTCTAGCTATTAT 3′; | (SEQ ID No.10) |
| 5′ ACAGTTAGCTAATGTTCAA 3′; | (SEQ ID No.11) |
| 5′ ACAAAGATCCTCCTAAATT 3′; | (SEQ ID No.12) |
| 5′ AAAGATCCTCCTAAATTGA 3′; | (SEQ ID No.13) |
| 5′ CTTGGCTGATCTCTATGAA 3′; | (SEQ ID No.14) |
| 5′ GGAGAGTGTTCTAGCTATT 3′; | (SEQ ID No.15) |
| 5′ GTAGAGCAATGGTAGATAT 3′; | (SEQ ID No.16) |
| 5′ GCTATTATCTCTTGTTACA 3′; | (SEQ ID No.17) |
| 5′ TAGAGCAATGGTAGATATA 3′; | (SEQ ID No.18) |
| 5′ GAUCUACCCUCCUGCUAUAUU 3′; | (SEQ ID No.19) |
| or | |
| 5′ AAACGAAUAUCGAAGAAUGUU 3′. | (SEQ ID No.20) |

31. The method according to claim 19 wherein the inhibitor is a neutralising antibody raised against MTBP or a fragment thereof.

32. The method according to claim 31 wherein the antibody is a polyclonal antibody.

33. The method according to claim 31 wherein the antibody is a monoclonal antibody.

34. The method according to 31 wherein the antibody is raised against the peptide CSSDWQEIHFDTE (SEQ ID No. 6).

35. The method according to 31 wherein the antibody is raised against the whole of MTBP.

36. The method according to claim 19 wherein the inhibitor is an inactive peptide fragment of MTBP that competes with endogenous MTBP and thereby reduces its activity.

37. The method according to claim 19 wherein the cancer is a cancer of the breast, bladder or lung.

38. The method according to claim 19 wherein the cancer is a cancer of mesothelial tissue.

39. The method according to claim 19 wherein the inhibitor is used in conjunction with another anti-cancer agent.
